(19) United States
(12) Patent Application Publication
(10) Pub. No.: US 2026/0272296 A1
(43) Pub. Date: Sep. 17, 2026

(54) Vision Testing Device with Interchangeable Lens Assembly Modules

(71) Applicant: Radius XR, LLC, Pleasanton, CA (US)

(72) Inventors: John Trefethen, Bellingham, WA (US); Thomas Nuzzo, San Francisco, CA (US); Chris Bradley, Baltimore, MD (US)

(21) Appl. No.: 19/551,513

(22) Filed: Feb. 26, 2026

Related U.S. Application Data
(60) Provisional application No. 63/765,103, filed on Feb. 28, 2025.

(51) Int. Cl. A61B 3/18; A61B 3/00; A61B 3/028; A61B 3/08; A61B 3/107; A61B 3/113

(52) U.S. Cl. CPC A61B 3/185; A61B 3/005; A61B 3/0285; A61B 3/08; A61B 3/107; A61B 3/113

(57) ABSTRACT

Head-mounted vision test devices include a host module with a processing unit and a display, and a lens assembly module detachably connectable to the host module. The host module automatically detects a type of the lens assembly module that is connected thereto. The lens assembly module includes an outer frame, at least one lens assembly comprising at least one lens adapted for testing at least one of visual field or visual acuity, and, preferably, at least one light block. Based on the detected type of the lens assembly module, the host module is configured to allow only one or more vision tests supported by the attached lens assembly module. The detachable lens assembly modules enable different vision tests to be performed using a single head-mounted device while preventing incompatible test selection and improving testing reliability.

### CROSS-REFERENCE

The present application relies on U.S. Patent Provisional Application No. 63/765,103, titled “Vision Testing Device with Interchangeable Lens Assembly Modules” and filed on Feb. 28, 2025, for priority, which is herein incorporated by reference in its entirety.

### FIELD

The present specification relates to vision assist and/or diagnostic systems and methods. Specifically, the embodiments disclosed herein describe use of a head-mounted vision device adapted to be coupled with one or more modular, detachable lens assemblies, wherein each lens assembly is designed to enable one or more diagnostic tests.

### BACKGROUND

In a conventional clinical setup, some visual acuity tests determine how well an individual can see a letter or symbol of a given size from a predefined distance. For example, in a Snellen test, letters are arranged in rows and/or columns. From row to row, the letters have different sizes, typically decreasing in size as one visually progresses from a higher row to a lower row. Standing 14 to 20 feet away, an individual attempts to identify each letter accurately. A value representative of the individual's visual acuity may be determined based on how far the individual is able to accurately progress in the chart (the smallest letter the individual is able to accurately identify). Alternatively, in a Random or Tumbling E test, an individual is presented a series of “E” images of different sizes and instructed to identify the direction each letter “E” is facing, such as up, down, left, or right. Again, a value representative of the individual's visual acuity may be determined based on how far the individual is able to accurately progress in the chart (the smallest letter the individual is able to accurately identify). Regardless of the type of visual acuity test being used, a conventional visual acuity test requires an individual to accurately identify the type of, or orientation of, differently sized letters, symbols, or figures, generally referred to as optotypes.

Also, conventionally, testing a user's visual field and/or peripheral vision is performed with dedicated devices and systems. The Humphrey visual field test (VFT) is a common peripheral vision test. Other visual field tests may include a confrontation visual field test, an automated static perimetry test, a kinetic visual field test, a frequency doubling perimetry test, and an electroretinography (ERG) test. For the automated static perimetry test, a user is provided with a device that displays visual stimuli (typically a small spot of light) at a predetermined set of test locations in the user's peripheral visual field. The user is instructed to fixate on a fixed target while detecting any spot of light that appears in the peripheral visual field. Typically, the spot of light is presented at each test location multiple times, and at different contrast levels. The contrast level at which the probability of detection is at a predetermined level (usually set to 50% probability) represents the contrast threshold at that test location. If the contrast threshold is sufficiently high (equivalently, contrast sensitivity is sufficiently low), the system determines that the user may have a deficit in his or her visual field at that test location. Stimulus presentation at any test location may be optimized to reduce the number of measurements by leveraging estimates of contrast thresholds at neighboring test locations, dynamically changing the time between stimulus presentations based on the distribution of user response times and leveraging normative databases of contrast sensitivity for different age groups. Examples of algorithms that implement such optimization procedures are SITA, ZEST, RATA, and SWeLZ. In certain cases, the VFT may be used to present the stimulus at only the highest possible contrast level as a fast screening method.

The Amsler Grid test is a tool for discovering a user's visual impairments and involves a user looking at a printed grid of squares to determine whether there are any perceived artifacts or irregularities in the grid. The grid structure used for the test is simple and the contents within the grid are usually known to the user, which makes it easier to observe and note any differences between the actual content and what is visually perceived. Describing abnormal vision can be performed on paper with a similar grid where a user can mark the areas of distortion for different kinds of distortions that may occur. Amsler grids are commonly 20×20 squares that may be placed at a distance to a user's eye such that each square subtends one degree of visual angle. Different versions of the grid may have varying features, such as color, for example. The Amsler Grid test is subjective; thus, a severity of vision impairment cannot easily be quantified through the test. The test is intended to be a quick and simple screening tool to determine common vision abnormalities such as, but not limited to scotoma, voids, holes, blind spots, and missing area in a user's vison.

Some of the current head-mounted ophthalmologic devices for performing such tests, may be limited to either high-resolution or wide field-of-view capabilities, due to the nature of the display and proximity to the patient's face, which can restrict their effectiveness in diagnosing or monitoring certain conditions. For instance, performing visual acuity tests via a head-mounted vision test device may require a very high pixel density to resolve the very small characters that are viewed within close proximity to the patient's face, while visual field tests for conditions such as glaucoma require a wide field of view. High resolution and a wide field of view (FOV) are typically mutually exclusive in head-mounted displays since increasing the FOV spreads the available pixels over a larger area, thereby reducing angular resolution. Traditional diagnostic devices may lack the capability of addressing the two competing needs in one device-both high-resolution and a wider field-of-view-meaning that clinicians must use multiple devices or settle for compromised testing accuracy.

Hence there is need for a vision testing device that provides systems and methods for vision testing that afford reduced variance and provide improved diagnostic results. There is also need for a single versatile vision test device that can perform different types of vision tests requiring both high-resolution or wide field-of-view capabilities, thereby optimizing testing procedures and broadening the range of possible diagnostics.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification is directed towards a head-mounted vision test device comprising: a host module, comprising a processing unit and a display; a lens assembly module, wherein the lens assembly module is adapted to be detachably connected to the host module and wherein the host module is configured to automatically detect and identify a type of a lens assembly module that is connected to the host module, wherein the lens assembly module comprises: an outer frame and at least one lens assembly comprising at least one lens, adapted for testing one of either a visual field of a patient or a visual acuity of the patient, and wherein the host module is configured to execute only one or more vision tests that are uniquely supported by the identified lens assembly module.

Optionally, the outer frame comprises a button which, when depressed, releases the lens assembly module from the host module.

Optionally, the outer frame comprises a label indicative of the type of the lens assembly module.

Optionally, the lens assembly further comprises at least one light block.

Optionally, the host module comprises a strap to fit around the patient's head while wearing the head-mounted vision test device.

Optionally, the strap includes a knob configured to be rotated to adjust a fit of the strap round the patient's head.

Optionally, an inner region of the outer frame comprises a first plurality of alignment guides to align with a corresponding second plurality of alignment guides in an inner region of the host module, and wherein the host module is attached to the lens assembly module using a plurality of magnets once the first plurality of alignment guides aligns with the corresponding second plurality of alignment guides.

Optionally, the host module comprises a first internal chassis including a first host module feature and a second internal chassis including a second host module feature, wherein the lens assembly module comprises a first lens assembly including a first lens feature and a second lens assembly including a second lens feature, and wherein the first host module feature is configured to receive the first lens feature and the second host module feature is configured to receive the second lens feature to enable the lens assembly to be detachably connected to the host module.

Optionally, the first and second internal chassis are covered by an outer housing, and wherein the outer housing includes a first glass layer and a second glass layer for respectively coupling with the first lens assembly and the second lens assembly.

Optionally, each of the first and second glass layers is fabricated from fusilica, gorilla glass, or BK7.

Optionally, the lens assembly module is configured for one of a central visual field test, a ptosis and binocular visual field test, a visual acuity test, a phoropter test or a keratoconus test.

Optionally, the lens assembly module is configured for a visual acuity and phoropter test, and wherein the lens assembly module comprises a plurality of lenses that provide a 14 degrees field of view.

Optionally, the lens assembly module is configured for a central visual field test including threshold and suprathreshold testing.

Optionally, the lens assembly module operates within a 60° field of view.

Optionally, the lens assembly module is configured for a central visual field test including monocular and binocular vision testing.

Optionally, the lens assembly module is configured for a ptosis and binocular visual field test.

Optionally, the lens assembly module operates within a 120° field of view.

Optionally, the lens assembly module comprises one or more pancake lenses.

Optionally, the lens assembly module is configured for a keratometry test by measuring corneal curvature of a patient.

Optionally, the head-mounted vision test device further comprises a remote control device tethered to the head-mounted vision test device.

Optionally, the remote control device comprises a battery for powering the head-mounted vision test device.

Optionally, the host module comprises means for tracking an eye movement of the user.

Optionally, the lens assembly module comprises optically distinct lens stacks configured to optimize at least one of a field of view, an angular resolution, a luminance transmission, and an optical magnification for a corresponding diagnosis. Optionally, each lens stack is structurally configured for a specific diagnostic test selected from visual acuity testing, visual field testing, binocular field assessment, or corneal curvature assessment.

Optionally, the host module is further configured to disable a selection of one or more diagnostic tests that are incompatible with optical characteristics of the identified lens assembly module.

In some embodiments, the present specification is directed towards a method of using a visual test device, comprising: determining at least one eye test for a patient; selecting, based on the determined at least one eye test, a first lens assembly module from a plurality of types of lens assembly modules; attaching the selected first lens assembly module to a host module to form the visual test device; automatically detecting, by the host module, a type of the first lens assembly module in order to determine a corresponding first eye test to be allowed and performed using the first lens assembly module; mounting the visual test device on the first patient's head; and using the visual test module to perform the first eye test on the first patient.

Optionally, the method further comprises detaching the first lens assembly module from the host module and attaching a second lens assembly module to the host module if an additional second eye test is needed by the first patient; automatically detecting, by the host module, a type of the second lens assembly module in order to determine a corresponding second eye test to be allowed and performed using the second lens assembly module; and using the visual test module to perform the second eye test on the first patient.

In some embodiments, the present specification is directed towards a method of using a visual test device, comprising: determining at least one eye test for a first patient and at least one eye test for a second patient; selecting a first lens assembly module, based on the determined at least one eye exam/test needed by the first patient, and a second lens assembly, based on the determined at least one eye exam/test needed by the second patient, from a plurality of types of lens assembly modules; attaching the selected first lens assembly module to a host module to form the visual test device; automatically detecting, by the host module, a type of the first lens assembly module in order to determine a corresponding first eye test to be allowed and performed using the first lens assembly module; mounting the visual test device on the first patient's head; using the visual test module to perform the first eye test on the first patient; removing the visual test device from the first patient's head, detaching the first lens assembly module from the host module and attaching the second lens assembly module to the host module; automatically detecting, by the host module, a type of the second lens assembly module in order to determine a corresponding second eye test to be allowed and performed using the second lens assembly module; mounting the visual test device on the second patient's head; and using the visual test module to perform the second eye test on the second patient. Optionally, the first eye test is different from the second eye test.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is an illustration of a host module and a lens assembly module of a vision testing device, in accordance with an embodiment of the present specification;

FIG. 1B is an illustration of the vision testing device of FIG. 1A, showing the lens assembly attached to the host module, in accordance with an embodiment of the present specification;

FIG. 1C illustrates another view of the vision testing device of FIGS. 1A and 1B in accordance with an embodiment of the present specification;

FIG. 1D is an illustration of a lens assembly module, in accordance with an embodiment of the present specification;

FIG. 1E is an illustration of the lens assembly module and alignment holes such that it can be aligned to the host module, in accordance with an embodiment of the present specification;

FIG. 1F is an illustration of the lens assembly module and various coupling points to the host module, in accordance with an embodiment of the present specification;

FIG. 1G is an exploded view illustration of the vision testing device, in accordance with an embodiment of the present specification;

FIG. 1H illustrates a housed internal chassis of the host module, in accordance with an embodiment of the present specification;

FIG. 2A illustrates a kit for a vision diagnostic system comprising multiple components integrated into a single contiguous container, in accordance with an embodiment of the present specification;

FIG. 2B illustrates a plurality of lens assembly modules of the vision testing device, in accordance with an embodiment of the present specification;

FIG. 3A illustrates a visual acuity test module coupled with the host module of the vision test device, in accordance with an embodiment of the present specification;

FIG. 3B illustrates a visual acuity test module and the host module of the vision test device, in accordance with an embodiment of the present specification;

FIG. 3C illustrates the visual acuity test module of FIGS. 3A and 3B, in accordance with an embodiment of the present specification;

FIG. 3D is a transparent view illustration of the visual acuity test module, in accordance with an embodiment of the present specification;

FIG. 3E illustrates various lenses that may be used with a lens assembly of the visual acuity test module, in accordance with an embodiment of the present specification;

FIG. 3F illustrates a width of the visual acuity test module, in accordance with an embodiment of the present specification;

FIG. 3G illustrates a diameter of the various lenses that may be used with the visual acuity test module, in accordance with an embodiment of the present specification;

FIG. 3H illustrates another view of the visual acuity test module attached to the host module of the vision test device, in accordance with an embodiment of the present specification;

FIG. 3I illustrates another view of the visual acuity test module, in accordance with an embodiment of the present specification;

FIG. 4A illustrates a central visual field test module coupled with the host module of the vision test device, in accordance with an embodiment of the present specification;

FIG. 4B illustrates a central visual field test module detached from the host module of the vision test device, in accordance with an embodiment of the present specification;

FIG. 4C illustrates another view of the central visual field test module, in accordance with an embodiment of the present specification;

FIG. 4D illustrates various lenses that may be used with a lens assembly of the central visual field test module, in accordance with an embodiment of the present specification;

FIG. 4E illustrates a diameter of the various lenses that may be used with the central visual field test module, in accordance with an embodiment of the present specification;

FIG. 4F illustrates yet another view of the central visual field test module coupled with the host module of the vision test device, in accordance with an embodiment of the present specification;

FIG. 5A illustrates a ptosis and binocular visual field test module coupled with the host module of the vision test device, in accordance with an embodiment of the present specification;

FIG. 5B illustrates another view of the ptosis and binocular visual field test module coupled with the host module of the vision test device as shown in FIG. 5A, in accordance with an embodiment of the present specification;

FIG. 5C illustrates a width of the ptosis and binocular visual field test module, in accordance with an embodiment of the present specification;

FIG. 5D illustrates lenses of a lens assembly of the ptosis and binocular visual field test module, in accordance with an embodiment of the present specification;

FIG. 5E illustrates a diameter of the lenses of the ptosis and binocular visual field test module, in accordance with an embodiment of the present specification;

FIG. 5F illustrates the ptosis and binocular visual field test module attached with the host module of the vision test device, in accordance with an embodiment of the present specification;

FIG. 5G illustrates the ptosis and binocular visual field test module detached from the host module of the vision test device, in accordance with an embodiment of the present specification;

FIG. 6 illustrates a lens assembly of the keratoconus test module in accordance with an embodiment of the present specification;

FIG. 7A illustrates a model of the vision test device in communication with a haptic control device, in accordance with an embodiment of the present specification;

FIG. 7B illustrates another model of the vision test device in communication with a haptic control device, in accordance with an embodiment of the present specification;

FIG. 7C illustrates yet another model of the vision test device in communication with a haptic control device, in accordance with an embodiment of the present specification;

FIG. 8A is a flow chart describing steps of a method for using the visual test device with at least one lens assembly module with one patient, in accordance with some embodiments of the present specification;

FIG. 8B is a flow chart describing steps of a method for using the visual test device with at least one lens assembly module switching from a first patient to a second patient, in accordance with some embodiments of the present specification;

FIG. 9A illustrates the host module/head mounted device (HMD) coupled with a strap, in accordance with an embodiment of the present specification;

FIG. 9B illustrates the strap being released from the body of the host module by pressing a button, in accordance with an embodiment of the present specification;

FIG. 9C illustrates a button, a spring, and a portion of the body of the host module that houses the button and the spring, in accordance with an embodiment of the present specification;

FIG. 9D illustrates a close-up view of the button and the portion of the body of the host module that houses the button, in accordance with an embodiment of the present specification;

FIG. 9E is a pictorial diagram showing a first side of the button, in accordance with an embodiment of the present specification;

FIG. 9F is a pictorial diagram showing a second side of the button, where the second side opposes the first side, in accordance with an embodiment of the present specification;

FIG. 9G illustrates an attachment portion of the strap, in accordance with an embodiment of the present specification;

FIG. 9H illustrates the attachment portion of the strap and a slot provided within the body of the host module for receiving the attachment portion, in accordance with an embodiment of the present specification;

FIG. 9I illustrates the attachment portion of the strap partially inserted into the slot provided in the body of the host module, in accordance with an embodiment of the present specification;

FIG. 9J is a side plan view of the strap attached to the body of the host module, in accordance with an embodiment of the present specification; and

FIG. 9K is a side plan view of the strap being released from the body of the host module, in accordance with an embodiment of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures. In an embodiment, the head mounted vision testing device as described in the present specification includes at least an internal general-purpose computer (GPU) configured for executing a plurality of vision testing software. In an embodiment, at least one WIFI-enabled computing device may be coupled with the vision testing device. In an embodiment, the at least one WI-FI computing devices includes eye exam software and is configured to be in communication with the head-mounted vision device to send eye exams/instructions to the patient and to receive data from the HMD. In another embodiment, the vision testing device includes a WIFI connection for supporting the communication of vision testing data with other remote computing platforms and databases.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application” or “engine” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

### Overview

The present specification provides a vision testing device comprising a host module (also referred to as a “lens holder”) which is configured to be coupled or attached to at least one of a plurality of detachable lens assemblies or modules such that a clinician may select lenses that align with specific testing needs, such as different fields of view, resolution levels or luminosity. In embodiments the host module is a head mounted device (HMD). In embodiments, the host module is configured to automatically detect which lens assembly or module is connected to the host module and activate only the tests that are relevant and enabled by the specific lens assembly that is coupled with the host module. In embodiments, the host module is designed as a wearable headset.

In some embodiments, upon identification of the attached lens assembly module 104, the host module 102 executes a test-gating protocol stored in memory, wherein only diagnostic tests compatible with the optical, field-of-view, resolution, and luminance characteristics of the identified module are enabled for selection. Tests incompatible with the identified module are automatically disabled or hidden from a user interface of the host module 102. In certain embodiments, the host module 102 further prevents initiation of an incompatible test even if manually selected, thereby ensuring that diagnostic procedures are constrained to the operational capabilities of the attached lens assembly module. In some embodiments, upon system startup, the attached lens assembly module is detected. If a user attempts to run a test that is not supported by the attached lens assembly module, the user will be prompted by the system to first secure the appropriate lens assembly module.

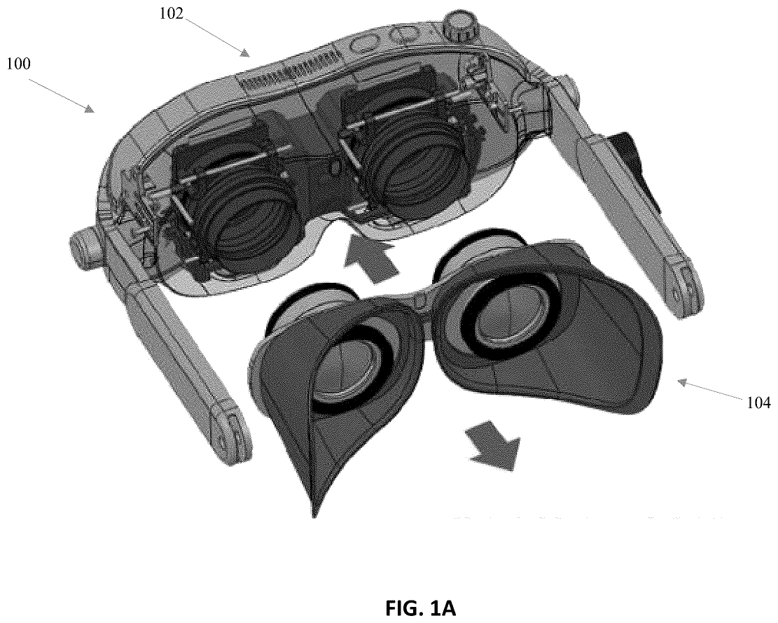

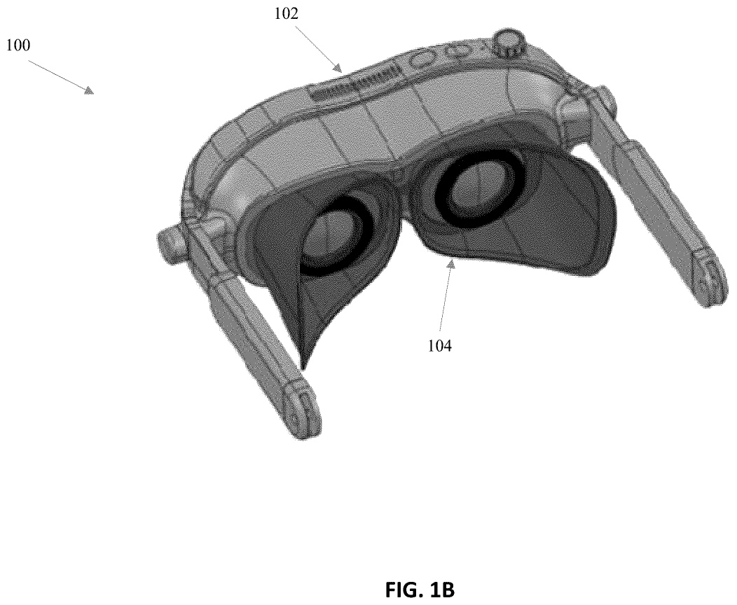

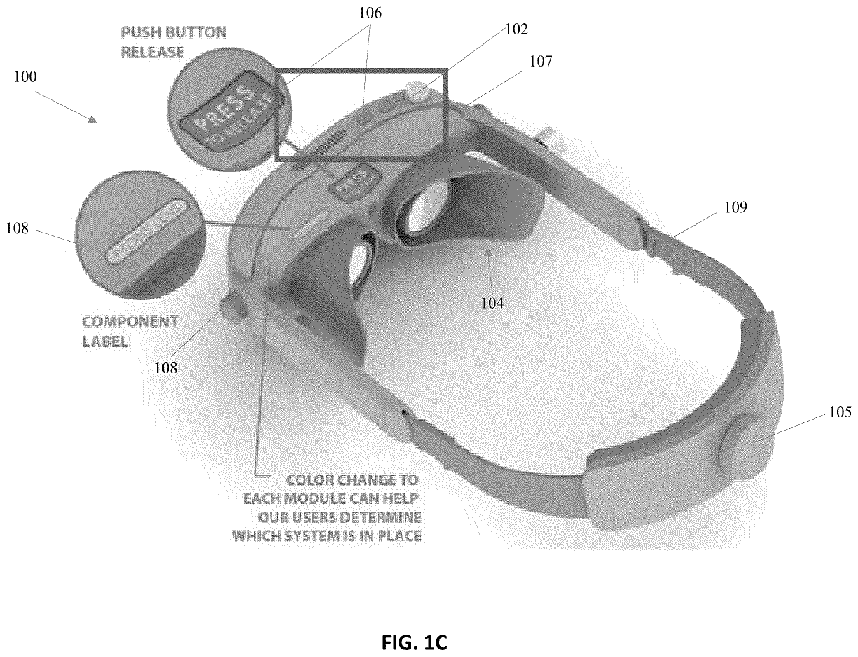

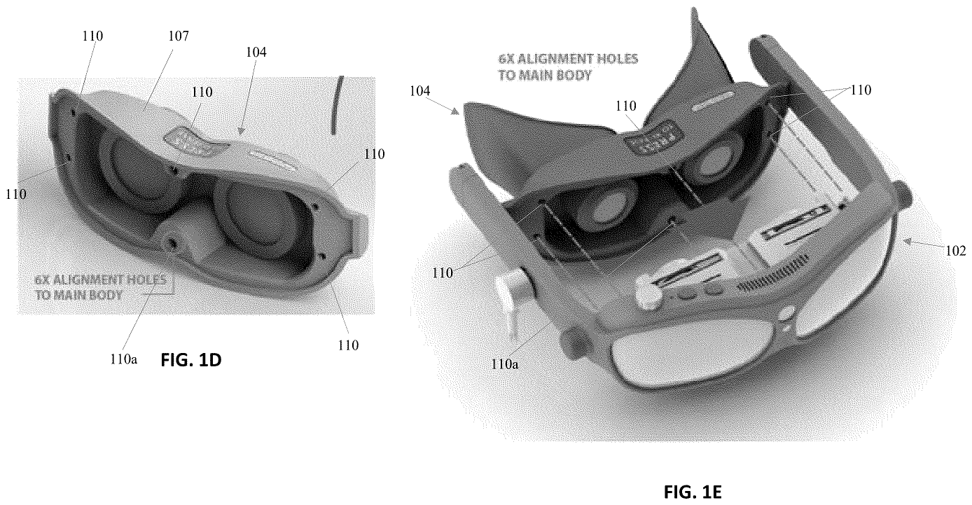

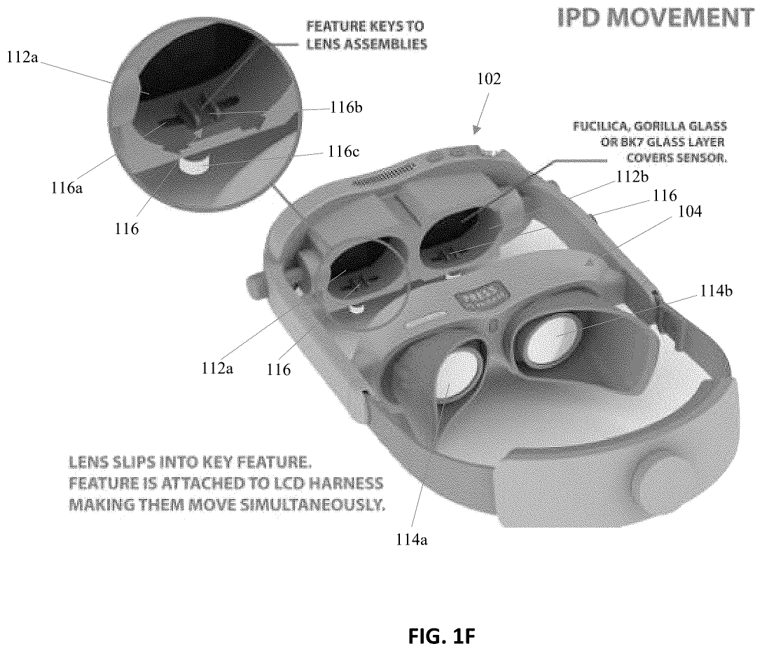

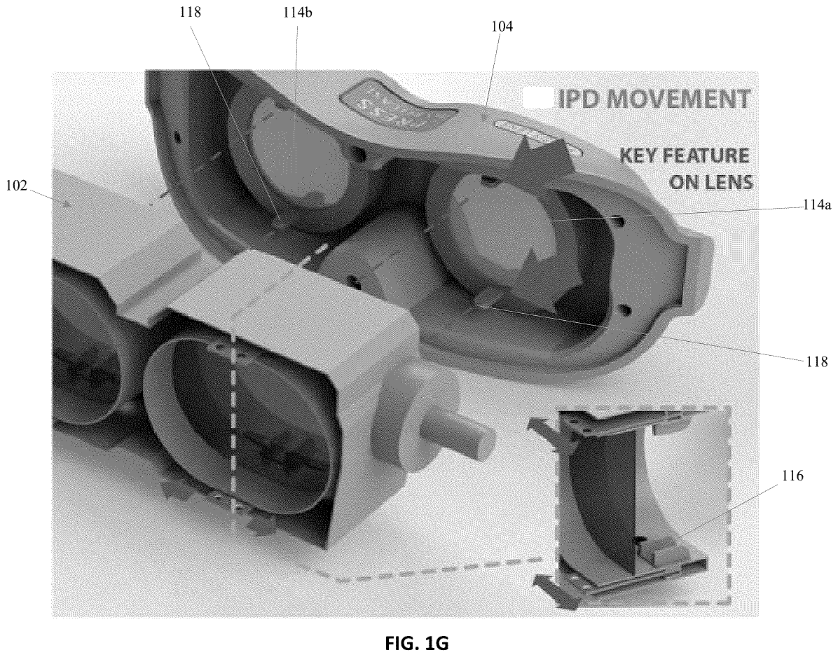

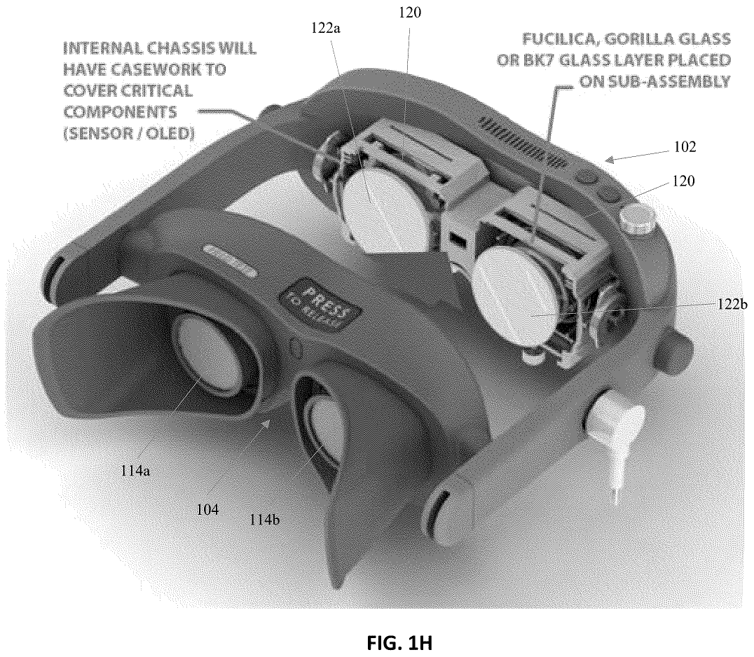

FIG. 1A illustrates a host module and a lens assembly module of a vision testing device, in accordance with an embodiment of the present specification. FIG. 1B illustrates the vision testing device, in accordance with an embodiment of the present specification. Referring to FIGS. 1A and 1B simultaneously, vision testing device 100 comprises a host module 102 which may be coupled with at least one of a plurality of different lens assembly modules such as lens assembly module 104. Lens assembly module 104 is configured to fit into the host module 102 enabling the vision testing device 100 to be used to perform one or more vision tests that are pre-defined and based on the parameters of the lens assembly module 104. In embodiments, and as described throughout the specification, lens assembly module 104 includes, among other components, an outer frame, dedicated lens assemblies and optics, which are described in greater detail below.

In embodiments, the host module 102 houses core processing, display, and detection components of the vision testing device of the present specification, thereby serving as a central platform and processor for all diagnostic activities. A plurality of interchangeable lens assembly modules 104 may be connected with the host module 102. Different lens assembly modules 104 have different field-of-view and resolution capabilities in order to cater to specific diagnostic needs and therefore are fitted with different lens assemblies and various other components that lend to the functionality. In various embodiments, the host module 102 is configured to detect the attached lens assembly module 104 type and automatically restricts available tests to those compatible with the module's capabilities, thereby preventing misuse, and enhancing test reliability. In an embodiment the lens assembly module 104 is coupled with the host module 102 via a USB Type-C connector or pogo pins. Upon connection with the host module 102, the lens assembly module 104 transmits an identification signal to the host module via the USB Type-C connector that allows the host module 102 to identify the lens assembly module 104 and restrict available tests to those compatible with the module's capabilities. In other embodiments, identification of the lens assembly module may be accomplished using passive identifiers, such as, but not limited to RFID, NFC, mechanical keying, and optical markers.

The host module 102 is designed as a headset to be worn by a user/patient undergoing vision testing, after a lens assembly module 104 has been attached to/fitted into/coupled with the unit, such as the vision testing device shown in FIG. 1B. The host module 102 is manufactured to feel like wearing a pair of glasses or goggles when in use by a patient.

In embodiments, the host module 102 is in data communication with a lens assembly module 104 once the lens assembly module 104 is coupled, attached or connected to the host module 102. In various embodiments, the data communication may be wired and/or wireless. In case of wireless data communication, the host module 102 and the lens assembly module 104 may use Bluetooth or any other wireless protocol known to persons of ordinary skill in the art.

FIG. 1C illustrates another view of the vision testing device in accordance with an embodiment of the present specification. As shown, the lens assembly module 104 is fitted into/attached to the host module 102. In embodiments, lens assembly module 104 comprises an outer frame 107. The outer frame 107 comprises a button 106 which, when depressed, supports the release or disconnect of the lens assembly module 104 from the host module 102. The outer frame 107 also comprises, in embodiments, a label 108 which describes the type/function of the lens assembly module 104. The label 108 enables a user to ascertain which type of lens assembly module corresponding to which type of vision test is connected to the host module 102. In other embodiments, a color of the outer frame 107 may be used to indicate the type of the lens assembly module. Different lens assembly modules having different functions may have frames of different colors in order to enable a user to visually recognize the type/functionality of the lens assembly module. In still other embodiments, the host module, which is configured to detect which lens assembly module is connected thereto, will display the type of lens assembly module that is connected, on an integrated display, and prompt the user to confirm that the correct module is connected. In embodiments, the host module 102 also comprises a strap 109 which is designed to fit around a patient's head when the patient is wearing the vision testing device 100, ensuring a snug fit of the device 100 around the patient's eyes. The strap 109 comprises a feature 105 which, in an embodiment, is a knob that may be rotated in order to adjust strap 109 such that it fits comfortably around the patient's head. In embodiments, the strap 109 is adjusted such that the headset does not move or change position during testing.

FIG. 1D illustrates a lens assembly module, in accordance with an embodiment of the present specification. FIG. 1E illustrates the lens assembly being coupled with the host module, in accordance with an embodiment of the present specification. Referring to both FIGS. 1D and 1E, in embodiments, the outer frame 107 of the lens assembly module 104 comprises a plurality of alignment guides/holes 110. As shown in FIGS. 1D and 1E, alignment guides/holes 110 are provided in an inner region of the frame 107 to align with corresponding alignment guides (not visible in FIG. 1E) in an inner region of the host module 102. In embodiments, six alignment guides/holes 110 are provided on the outer frame 107 of the lens assembly module with a corresponding number provided on the host module. In other embodiments, the frame 107 and the host module 102 comprise magnetic elements (in corresponding numbers) that enable the frame 107 to magnetically be attached to the host module 102. In embodiments, the central hole/alignment guide 110a comprises a USB Type-C connector. Upon connection with the host module 102, the lens assembly module 104 transmits an identification signal to the host module via the USB Type-C connector that allows the host module 102 to identify the lens assembly module 104 and restrict available tests to those compatible with the module's capabilities.

In an embodiment, eye tracking of a patient may be conducted via either the host module or the lens assembly module. Eye-tracking systems generally determine a user's gaze direction by optically sensing features of one or both eyes and computationally estimating a corresponding line of sight, and such systems may be categorized into several technical approaches with differing hardware configurations and processing methodologies. A prevalent class of systems employs video-oculography (VOG), in which one or more image sensors, integrated into at least one of the host and lens assembly module, capture images of the eye illuminated by visible or near-infrared light sources, often arranged to generate corneal reflections (glints) on the anterior surface of the eye. Image processing algorithms, which may be local to the device or remotely executing in a server, identify features such as the pupil boundary, pupil center, iris contours, eyelid margins, and one or more glints, and derive gaze direction based on geometric relationships between the detected pupil and the corneal reflections, optionally using calibration-based regression models or three-dimensional eye models to map image-space features to a gaze vector. Variations of VOG systems include dark-pupil and bright-pupil techniques, depending on the relative alignment of the illumination source and the image sensor, as well as single-camera, multi-camera, or stereo-camera configurations that enhance robustness to occlusion and improve estimation of eyeball center and corneal curvature.

Other eye-tracking approaches include electro-oculography (EOG), which measures corneo-retinal standing potentials via electrodes positioned around the eye socket of the lens assembly or host module to infer eye rotation from voltage differentials; scleral search coil systems, which detect induced voltages in a coil embedded in a contact lens placed on the eye within a magnetic field to provide highly precise measurements of ocular orientation; and limbus-tracking systems that detect contrast between the sclera and iris at the limbus boundary to determine eye position.

Additionally, structured light or time-of-flight depth sensing may be incorporated into at least one of the host and lens assembly module to obtain three-dimensional measurements of eye geometry, and may utilize machine learning models trained on labeled gaze data to directly regress from image features to gaze direction without explicit geometric modeling. Eye-tracking systems may further include calibration procedures to compensate for individual anatomical differences, such as angle differences between optical and visual axes, headset positioning variability, or camera alignment tolerances, and may employ real-time filtering, prediction, and drift correction algorithms to maintain stable gaze estimation under conditions of head motion, blink events, and variable illumination.

FIG. 1F illustrates another view of the vision testing device, in accordance with an embodiment of the present specification. FIG. 1G illustrates a close up view of the vision testing device, in accordance with an embodiment of the present specification. Referring to FIGS. 1F and 1G simultaneously, the host module 102 comprises a first internal chassis 112a and a second internal chassis 112b corresponding to and aligned with a first lens assembly 114a and a second lens assembly 114b, respectively, of the lens assembly module 104. In an embodiment, each of the first internal chassis 112a and second internal chassis 112b comprises a key feature 116 on the host module 102, and each of the first lens assembly 114a and second lens assembly 114b comprise a key feature 118, which enables each of the first lens assembly 114a and second lens assembly 114b to slide or slip into the each of the first internal chassis 112a and second internal chassis 112b enabling a connection between the host module 102 and the assembly module 104. As illustrated in FIG. 1G, lens features 118 refers to a structural engagement element formed on each lens assembly (lens assemblies 114a and 114b) of lens assembly module 104. Each lens feature 118 is configured to mechanically interface with a corresponding host module feature (key feature 116) provided on an internal chassis of the host module 102. Therefore, the engagement between lens feature 118 and host module feature 116 forms a mechanical interlock, wherein complementary structural elements of the lens feature and host module feature physically engage to resist separation in at least one direction without deformation of one or more components. In embodiments, lens feature 118 comprises a tab, rail, protrusion, flange, or keyed projection sized and shaped to slide into, seat within, or otherwise engage a corresponding slot, channel, or receptacle of the host module feature 116, thereby enabling detachable attachment of the lens assembly module 104 to the host module 102. In certain embodiments, the lens feature 118 is configured to translate laterally relative to the host module 102 in response to inter-pupillary distance (IPD) adjustment of the host module 102, while maintaining optical alignment between the lens assembly 114 and a corresponding display 112 of the host module. In one embodiment, the host module 102 uses a single contiguous display extending across both eyes while in another embodiment, the host module 102 comprises separate, distinct, independent displays positioned within each internal chassis 112a, 112b. The lens feature 118 may further provide positional stability, repeatable alignment, and load transfer between the lens assembly module 104 and the host module 102 during use. Key features 116, located on host module 102, are attached to a micro-OLED harness included in host module 102. The lens assembly module 104 is attached to the host module 102 by sliding each lens features 118 into each corresponding key feature 116 which is attached to a micro-OLED harness of the host module 102.

In various embodiments, the key features 116 are mechanically coupled to a micro-OLED harness of the host module 102 such that translational adjustment of the key features 116 for IPD adjustment correspondingly repositions micro-OLED display elements relative to the first and second lens assemblies 114a, 114b. In this manner, the optical axis of each display is maintained in alignment with a corresponding lens assembly during IPD adjustment while preserving the mechanical interlock formed between lens features 118 and host module features 116. Accordingly, the micro-OLED harness, key features 116, and lens features 118 collectively form an adjustable optical coupling interface between the host module 102 and the lens assembly module 104.

In one embodiment, each lens assembly 114a, 114b of lens assembly module 104 may be adjustable relative to host module 102. For example, the lens assemblies 114a, 114b may be configured to translate axially, laterally, or tilt relative to internal chassis 112a, 112b in order to adjust focus, spacing, optical alignment, or vergence. Such adjustment may be accomplished through mechanical interfaces associated with lens features 118 and host module features 116, including threaded engagement, sliding rails, cam mechanisms, micro-actuators, or other controlled translation mechanisms, while maintaining the mechanical interlock between lens feature 118 and host module feature 116. In certain embodiments, adjustment of lens assemblies 114a, 114b may be manual or electronically controlled by processing circuitry of host module 102.

In another embodiment, lens assemblies 114a, 114b are fixed relative to outer frame 107 and/or lens assembly module 104, such that optical spacing, tilt, and alignment are predetermined during manufacturing. In such embodiments, lens features 118 mechanically interlock with corresponding host module features 116 solely to secure the lens assembly module 104 to host module 102 without providing additional adjustment beyond inter-pupillary distance (IPD) movement enabled by host module features 116. Fixed configurations may provide enhanced structural rigidity and repeatable optical alignment during repeated attachment and detachment.

In an embodiment, key features 116 are configured to enable inter-pupillary distance (IPD) adjustment for the vision testing device, wherein the key features 116 each comprise both a slot 116a and a slidable tab 116b protruding above the slot, the tab being connected to a knob 116c protruding below the slot, such as shown in FIG. 1F. In embodiments, the knobs 116c may be rotated clockwise or counterclockwise by a user, resulting in translational motion of the tabs 116b in either direction, for adjusting the IPD of the vision testing device 100. As mentioned above, in embodiments, the tab 116b comprises two brackets with a space therebetween enabling key feature 118 of lens assembly 114a, 114b to slide into the space between the two brackets. Once the lens assembly module 104 is attached to the host module 102 by sliding each lens features 118 into corresponding each key features 116 which is attached to the micro-OLED harness of the host module 102, both the knobs 116c (one corresponding to each internal chassis 112a, 112b) may be adjusted simultaneously, by rotating either knob, thereby adjusting the IPD of the vision testing device.

FIG. 1H illustrates a housed internal chassis of the host module 102 in accordance with an embodiment of the present specification. The host module 102 comprises an outer housing/casework 120 for covering each internal chassis (not visible in FIG. 1H) which is configured to receive the first lens assembly 114a and second lens assembly 114b. The housing/casework 120 is also coupled with a first glass layer 122a and a second glass layer 122b, which are coupled with the first lens assembly 114a and second lens assembly 114b, respectively, when the lens assembly module 104 is coupled with the host module 102. In embodiments, each glass layer 122a and 122b may be fabricated from fusilica (fused silica), gorilla glass, or BK7 glass or any other material suitable for optics.

In an embodiment, a control/input device is tethered to the host module 102, allowing a user to interact with the host module 102 and also providing power (via a battery pack) to the host module 102. In embodiments, the host module 102 comprises integrated circuits (such as, but not limited to XR2 chipset), firmware and software along with a cooling mechanism, enabling functioning of the head mounted vision testing device of the present specification.

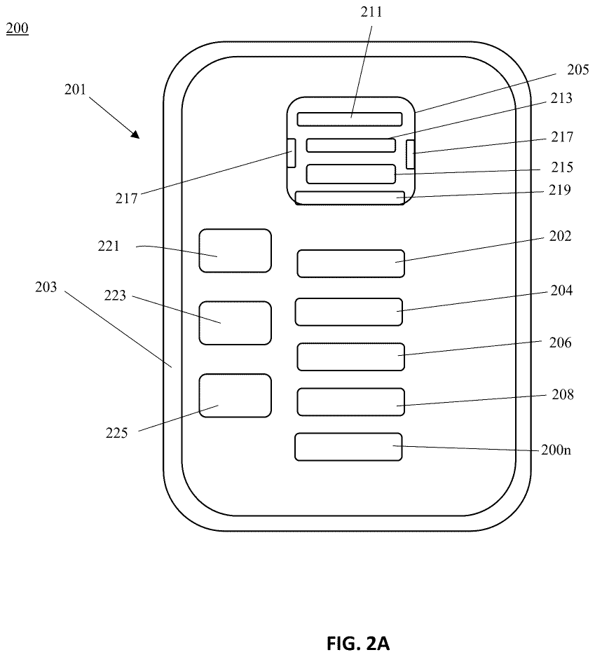

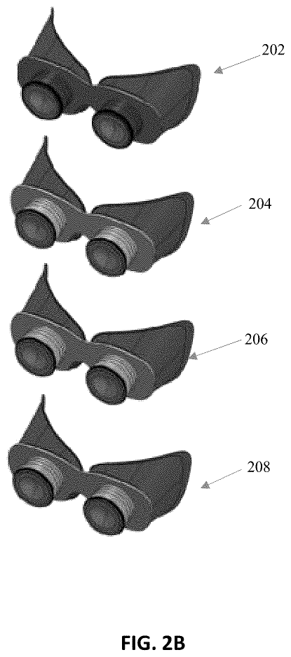

FIG. 2 illustrates a plurality of lens assembly modules that may be used with the vision testing device, in accordance with an embodiment of the present specification. In embodiments, each lens assembly module 202, 204, 206 and 208 is configured with precise optical characteristics suited to specific diagnostic tests. In various embodiments, for each lens assembly module, corresponding elements such as, but not limited to the lens assembly, sensor, and the micro-OLED harness are configured and arranged to perform or conduct the predefined vision tests that correspond to that module. Further, it should be noted that different components and parameters can be mixed and matched as needed to perform or conduct other vision tests that may not be described in the present specification.

In certain non-limiting embodiments, lens assembly modules may operate within example ranges such as:
a) a Field of View (FOV) of a lens assembly module used in the vision testing device is defined as: low when the FOV ranges from 14°-28°; mid-sized when the FOV ranges from 60°-80°; and high when the FOV ranges from 110° to 130° where, in each case, the angle refers to the angular field of view defined as the angular span from one edge of the captured image to the opposite edge;
b) a resolution (expressed as minimum resolvable angle (MAR) or the smallest angular separation between two features that can still be distinguished) of a lens assembly module used in the vision testing device is defined as: low when the resolution ranges from 5 to 6 arcminutes or higher; medium when the resolution ranges from 3 to 4 arcminutes; and high when the resolution ranges from 1 to 2 arcminutes or lower; and
c) a brightness of a lens assembly module used in the vision testing device is defined as: low when the brightness ranges from 80-120 cd/m² (candelas per square meter); medium when the brightness ranges from 300-1000 cd/m²; and high when the brightness ranges from 2500 cd/m² or higher.

In embodiments, the different lens assembly modules that may be attached to the host module in order to perform vision tests on a patient comprise a central visual field test module 202, a ptosis and binocular visual field test module 204, a visual acuity and phoropter module 206, and a keratoconus module 208, each having a dedicated lens assembly for performing the indicated tests. In certain embodiments, the host module may include a lens assembly identification and validation subsystem configured to ensure that a selected or inserted lens assembly corresponds to the requirements of a selected vision test protocol prior to initiation of the test. Each interchangeable lens assembly may include a machine-readable identifier, such as a passive or active RFID tag, NFC element, EEPROM, one-wire memory device, optical code, magnetic signature, or mechanical keying structure, storing data representative of one or more lens parameters including field of view, angular resolution, luminance capability, spectral transmission characteristics, distortion profile, or other optical performance attributes. The host module may include a corresponding reader or sensing interface positioned within the lens mounting interface to automatically detect the presence and identity of the inserted lens assembly upon installation. A processing unit within the host module may access a stored database or lookup table associating each vision test type with required or permissible lens parameter ranges, and may execute a validation routine that compares the detected lens assembly parameters with the parameter set required for the selected diagnostic test. If the detected lens assembly satisfies the predefined criteria (e.g., resolution within a required arcminute range and brightness capability exceeding a specified luminance threshold), the host module may enable the test to proceed. If the parameters do not align, the system may generate a user notification, inhibit test execution, or prompt insertion of a compatible lens assembly. In some embodiments, the validation routine may further verify firmware compatibility, calibration data integrity, or lens-specific distortion correction profiles prior to authorizing the test, thereby reducing the risk of diagnostic error attributable to use of an incompatible optical module.

In certain embodiments, if the validation routine determines that the inserted lens assembly fails compatibility, alignment, calibration, or performance threshold criteria associated with a selected visual field test, the host module may automatically transition into a restricted operating state in which execution of the visual field test protocol is prevented or conditionally limited. For example, the processing unit may disable initiation of stimulus presentation, suppress generation of test patterns beyond a predefined angular extent, or block progression past a pre-test calibration stage until corrective action is taken. In some implementations, the host module may perform an active alignment verification procedure prior to test authorization, including presenting one or more internal reference patterns, capturing eye-tracking data, assessing optical centering, detecting vignetting or field clipping, evaluating measured luminance against stored calibration values, or comparing measured distortion characteristics to a lens-specific distortion profile stored in memory. If the host module determines that the optical axis of the lens assembly is misaligned relative to the display subsystem, that measured luminance falls below a required threshold for the selected visual field protocol, or that the effective field of view does not meet minimum angular requirements, the host module may inhibit peripheral stimulus generation, restrict testing to a reduced central angular range, flag the test session as invalid, or prevent data recording. In further embodiments, the host module may generate an error code, log the incompatibility event in non-volatile memory, transmit a compatibility status to an external computing device, and/or display graphical instructions guiding a practitioner to reseat, replace, or recalibrate the lens assembly. In safety-critical diagnostic configurations, the host module may require successful completion of both lens identification validation and optical alignment confirmation before enabling full-intensity or high-brightness stimulus output, thereby preventing erroneous diagnostic results attributable to incorrect lens selection, improper seating, firmware mismatch, or degradation of optical performance.

In certain embodiments, referring to FIG. 2A, the vision diagnostic system 200 may be provided as a kit 201 where all of the following components are integrated into a single contiguous container 203. The container 203 comprises a headset main body 205 and a plurality of interchangeable lens assembly modules 202, 204, 206, 208 configured to selectively couple to the headset main body 205 to perform different diagnostic procedures. The headset main body 205 may include a display subsystem 211, processing circuitry 213, eye-tracking sensors 215, user interface components 217, and a mechanical and/or electrical coupling interface 219 adapted to removably receive one of multiple lens assembly modules 202, 204, 206, 208. Referring to FIGS. 2A and 2B simultaneously, the kit 201 may include, by way of example, a central visual field test module 202 configured to present stimuli within a defined central angular range; a ptosis and binocular visual field test module 204 configured to evaluate upper eyelid obstruction and coordinated binocular field performance; a visual acuity and phoropter module 206 configured to present optotypes and adjustable refractive stimuli for monocular and binocular acuity assessment; and a keratoconus module 208 configured to facilitate corneal irregularity screening through structured light presentation, distortion analysis, or other cornea-sensitive measurement techniques. Each module may comprise a distinct optical configuration, including selected combinations of field of view, angular resolution, luminance capability, spectral filtering, distortion characteristics, and/or embedded electronics, tailored to its associated diagnostic function. The modules 202, 204, 206, 208 may be packaged together with the headset main body 205 in a common housing, case, or commercial kit 201, optionally including calibration targets 221, hygienic interface components 223, and instructions for use 225, thereby enabling a practitioner to perform multiple clinically distinct vision assessments using a single adaptable platform. In some implementations, the kit 201 architecture permits expansion through the addition of future lens assemblies 200n compatible with the headset same main body 205, allowing the system 200 to support evolving diagnostic protocols without replacement of the core headset hardware.

Each test module has the functional role of a lens assembly module, with a specific test function. For example, in some embodiments, the central visual field test module 202 includes a lens assembly and other components that support a mid-sized field of view in a range of 50 to 90 degrees with mid brightness level. In preferred embodiments, the central visual field test module 202 includes a lens assembly and other components that support a mid-sized field of view of 70 degrees with mid brightness level. In embodiments, the ptosis and binocular visual field test module 204 includes a lens assembly and other components that support a wider field of view (greater than 110 degrees) but has a lower resolution as compared to the other modules. In embodiments, the visual acuity and phoropter module 206 includes a lens assembly and other components that has the highest resolution—1 minute or arc (which is the maximum and corresponds to 20/20 acuity)—and minifies objects/letters or anything within the visual field to a greater extent compared to the other lens assemblies. In embodiments, keratoconus module 208 includes a lens assembly and other components.

In some embodiments, the central visual field test module 202 comprises a Fresnel lens and allows 70% to 100% light transmission as the cost of form factor. In preferred embodiments, the central visual field test module 202 comprises a Fresnel lens and allows 98% light transmission as the cost of form factor. In some embodiments, the central visual field test module 202 may comprise at least one pancake lens. In embodiments, the ptosis and binocular visual field test module 204 comprises a Pancake lens, providing a wider field of view and a narrower form factor at the cost of reduced light transmission. In embodiments, visual acuity and phoropter module 206 comprises a lens. In embodiments, keratoconus module 208 comprises a lens. Listed below are some parameters of the different lens assembly modules that are non-limiting examples of those used with the vision test device of the present specification:

In one embodiment, the central visual field test module has a minimum field of view of 70 degrees, a minimum resolution of 0.43 deg for Nyquist frequency, a Fresnel or pancake lens type, 2-4 lenses, a brightness of 90% transmission from a 3000 cd/m² or nits display (2700 cd/m² or nits), and approximately 26 arcmin for Nyquist criteria. In one embodiment, the ptosis and binocular visual field test module has a minimum field of view of 110 degrees, a minimum resolution of 0.43 deg for Nyquist frequency, a pancake lens type, 2-4 lenses, a lens stack length in a range of 1 to 1.6 inches (preferably 1.3 inches), a brightness of 10% transmission from a 3000 cd/m² or nits display (300 cd/m²), and approximately 26 arcmin for Nyquist criteria. In one embodiment, the visual acuity and phoropter visual test module has a field of view in a range of 14-28 degrees, a minimum resolution of 1 arcminute (for 20/20 acuity), 3-6 lenses (preferably 5), a lens stack length in a range of 3 to 5 inches (preferably 4 inches), and approximately 1 arcmin for 20/20 acuity. In one embodiment, the keratoconus visual test module has a field of view in a range of 100-140 degrees (preferably 120 degrees).

In embodiments, each lens assembly module may be fabricated in a different color to differentiate the functionality of each module, so that a clinician can visually recognize which module to use. In embodiments, the modules can be easily attached to and detached from the host module of the vision testing device, allowing clinicians to swap the lens assembly modules as needed for different types of vision testing. Some exemplary lens assembly modules are described in greater detail below.

### Visual Acuity Test Module

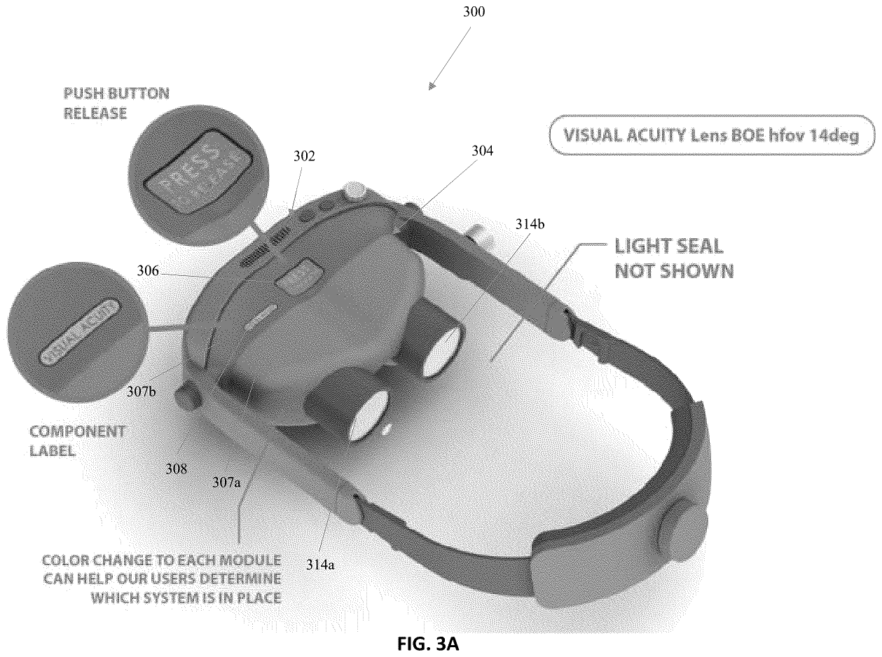

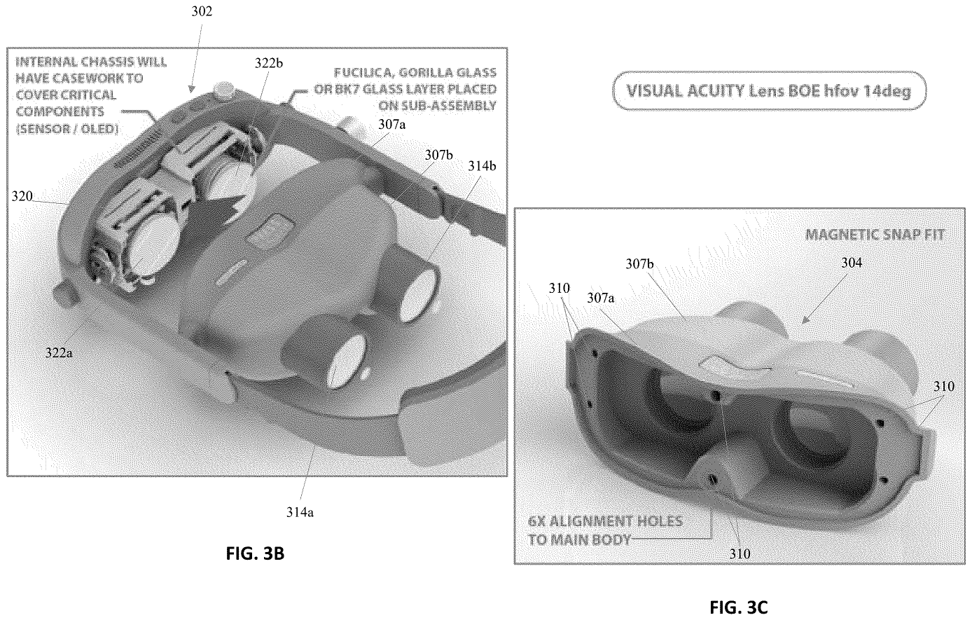

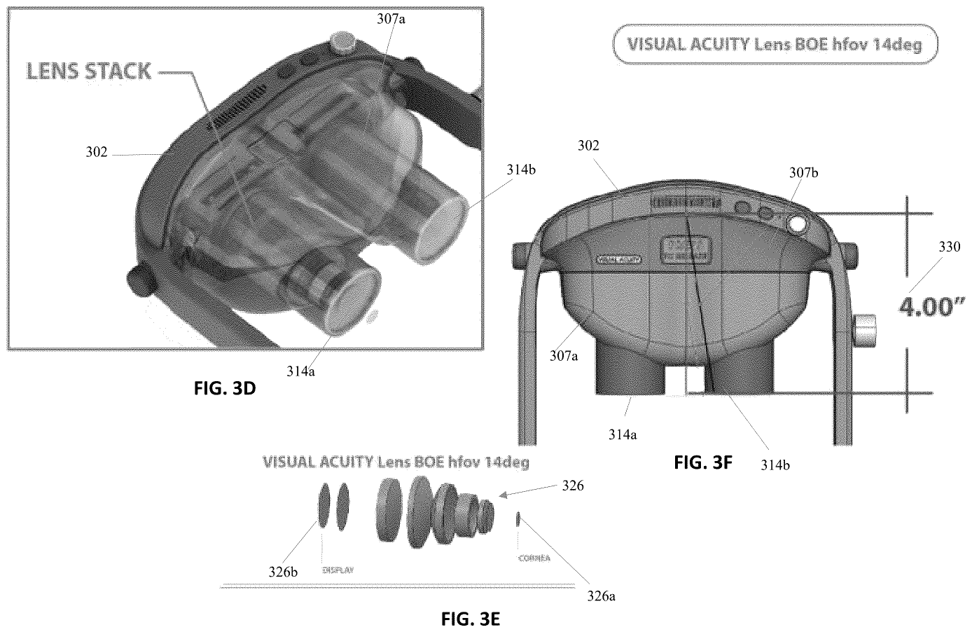

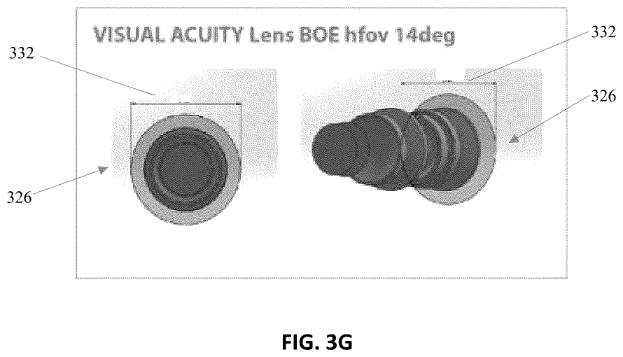

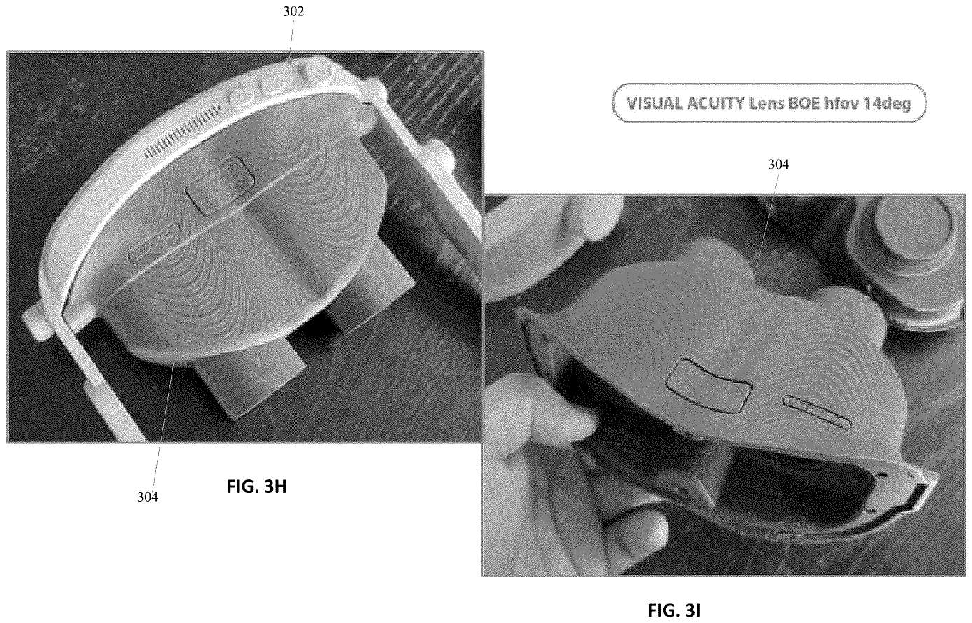

FIG. 3A illustrates a visual acuity test module 304, configured to measure the visual acuity of a patient, coupled with the host module 302 of the vision test device 300, in accordance with an embodiment of the present specification. FIG. 3B illustrates a visual acuity lens assembly module 304 and the host module 302, in a detached configuration, of the vision test device, in accordance with an embodiment of the present specification. FIG. 3C illustrates host module 302 of visual acuity test module 300, in accordance with an embodiment of the present specification. FIG. 3D is a transparent view illustration of the visual acuity test module 304, coupled with the host module 302 in accordance with an embodiment of the present specification. FIG. 3E illustrates a plurality of lenses of a lens assembly of the visual acuity test module, in accordance with an embodiment of the present specification.

In an embodiment, the visual acuity test module 304 has a restricted field of view (below 28°), enabling high-resolution testing which is required for detailed visual acuity assessments, such as up to 20/20 vision in patients. In embodiments, the visual acuity test module 304 is configured by integrating phoropter functionality to support further vision correction adjustments. Phoropter functionality includes measuring the eye's refractive error. In an embodiment, phoropter functionality affords testing of a patient's subjective refractive error, by using the auto-refractor's pre-set measure of the vision testing device, within a small, confined space, without the need for a larger room. In an embodiment, the lens and display parameters of the visual acuity test module 304 may be modified for testing patients that do not require 20/20 vision acuity measurement.

Referring to FIGS. 3A, 3B, 3C, 3D and 3E simultaneously, the visual acuity test module 304 comprises a first lens assembly 314a and a second lens assembly 314b. In embodiments, first lens assembly 314a and second lens assembly 314b are housed within an outer frame comprising a proximal portion 307a for protecting a plurality of lenses (not visible in FIG. 3A) of each lens assembly and a distal portion 307b which is adapted to attach to the host module 302. The distal portion 307b of the frame comprises a button 306 which, when pressed, enables the visual acuity test module 304 to be released or disconnected from the host module 302. The distal portion 307b also comprises a label 308 identifying the type/function of the module 304, which in this embodiment is “visual acuity”. In addition, the visual acuity test module 304 includes a light seal (not shown) for blocking out any light from outside of the device.

In embodiments, host module 302 comprises outer housing/casework 320 for covering at least one internal chassis (not visible in FIG. 3B), and preferably a corresponding pair configured to receive each of the first lens assembly 314a and second lens assembly 314b. The housing/casework 320 is also coupled with a first glass layer 322a and a second glass layer 322b, which are coupled with first lens assembly 314a and second lens assembly 314b, respectively, when the visual acuity test lens assembly module 304 is coupled with the host module 302. The distal portion 307b of the outer frame, which is adapted to attach with the host module 302 comprises six alignment holes/alignment guides 310 provided in an inner region to align with corresponding alignment guides in an inner region of the host module 302. In embodiments, the alignment guides 310 as well as the corresponding alignment guides in an inner region of the host module 302 comprise magnets having opposing polarities, thereby causing the host module 302 and lens assembly module 304 to be attached due to the magnetic force, once aligned and the appropriate identification of the connected module via the pogo pins or USB Type-C connection.

As shown in FIG. 3D, the proximal portion 307a of the outer frame covers each of the first lens assembly 314a and second lens assembly 314b. In an embodiment, each of the first lens assembly 314a and second lens assembly 314b comprise a plurality of lenses 326, shown in FIG. 3E with reference to a cornea of a patient at a proximal end 326a and a display positioned at a distal end 326b, wherein the display is placed proximate to each chassis in the host module 302.

In some embodiments the module includes movable internal lenses to change focus/diopter. Accordingly, in an embodiment, the visual acuity test module 304 includes one or more movable lenses within each of the first lens assembly 314a and second lens assembly 314b to enable adjustable optical power for vision correction. For example, one or more of lenses 326 (see FIG. 3E) within the lens stack may be configured to translate along an optical axis between the proximal end 326a and distal end 326b to vary focal length and diopter. Such translation may be achieved through internal sliding carriers, threaded interfaces, cam mechanisms, or electronically actuated micro-adjustment elements integrated within the visual acuity test module 304. In certain embodiments, movement of the lenses 326 is controlled manually or via processing circuitry of the host module 302 to support subjective refraction and phoropter-style testing.

In another embodiment, the lenses 326 of the first and second lens assemblies 314a, 314b are fixed relative to the outer frame portions 307a and 307b, and refractive correction is achieved through digital manipulation of images rendered by the display of the host module 302. In such embodiments, processing circuitry of the host module 302 modifies visual stimuli to simulate changes in spherical power, cylindrical power, axis, prism, or other refractive parameters. Digital adjustment may include controlled image scaling, blur simulation, distortion mapping, phase modulation, or contrast adjustment to support vision correction testing without physical movement of the lenses 326.

FIG. 3F illustrates a width 330 of the visual acuity test device 300, in accordance with an embodiment of the present specification. In some embodiment, the width 330 of the visual acuity test device 300 is approximately three to five inches from a center point on the device from either side. In a preferred embodiment, the width 330 of the visual acuity test device 300 is approximately four inches from a center point on the device from either side. FIG. 3G illustrates an overall diameter of the plurality of lenses 326, in accordance with an embodiment of the present specification. In embodiments, the plurality of lenses 326 provide a field of view ranging from 14 degrees to 28 degrees, and fulfills the outer diameter requirement related to an eye tracker, having a diameter 332 of 50 mm when viewed from both the front and side. Here, the outer diameter requirement refers to the requirements of standard commercial eye-tracking hardware specifications.

In one embodiment, an eye tracker is a wearable eye tracker having a front-scene camera that records what the user is looking at, thus allowing for first-person insights in real-world environments. Dimensions of an eye box used in a preferred eye tracker, comprise a width of 24-40 mm (preferably around 32 mm), a height of 20 to 32 mm (preferably around 26 mm), a radius of curvature of 6 to 20 mm (preferably around 13 mm), and a depth of 10 to 20 mm (preferably around 15 mm). In embodiments, the lens used in the vision test device of the present specification has a maximum lens outer diameter of 50 mm.

FIG. 3H illustrates another view of the visual acuity test module 304 fitted to the host module 302 of the vision test device 300, in accordance with an embodiment of the present specification. FIG. 3I illustrates another view of the visual acuity test module 304, in accordance with an embodiment of the present specification.

In some embodiments, a Subjective Refraction Test Module may be designed for connecting with the vision test device, by modifying the visual acuity test module 304 to include an aperture with two pin holes in the module design. In an embodiment, an LCD shutter is used to create the pinholes that can be varied in size, location orientation and opacity.

### Central Visual Field Test Module

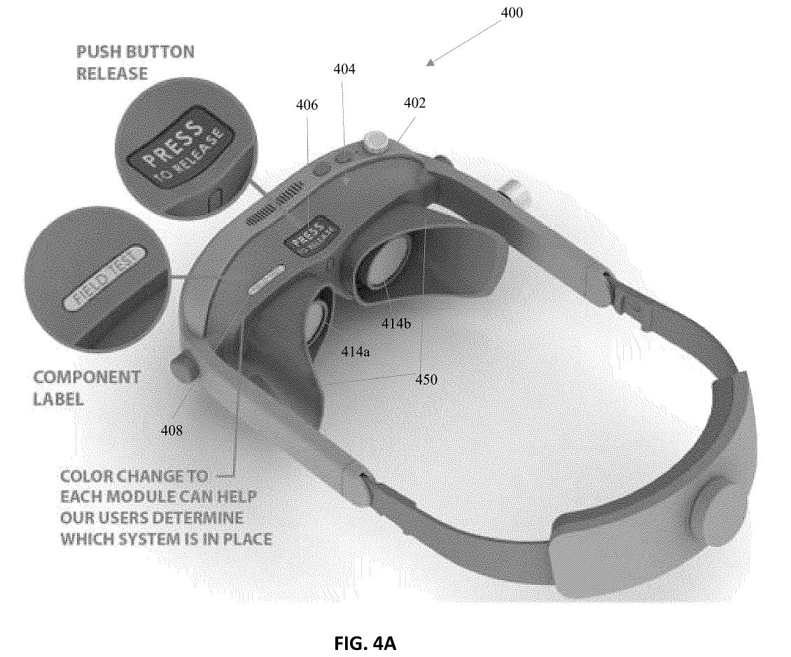

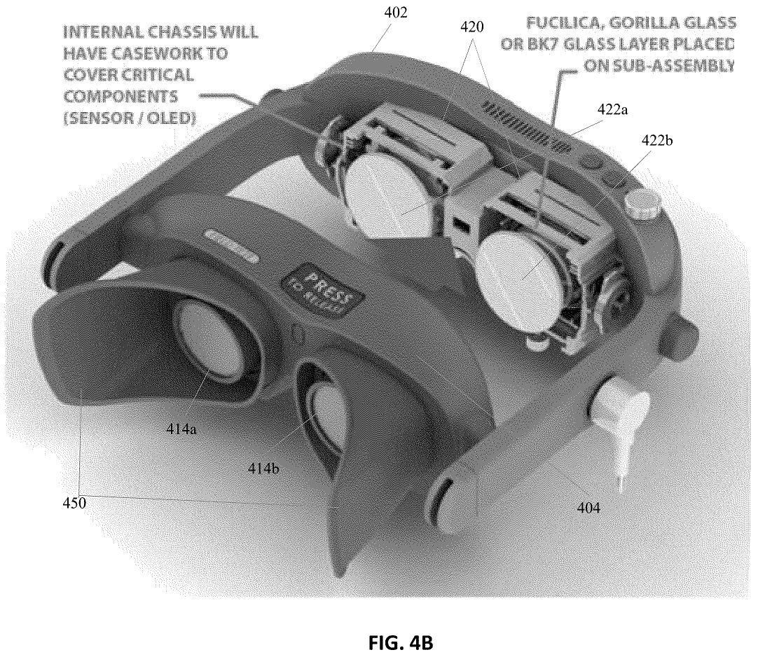

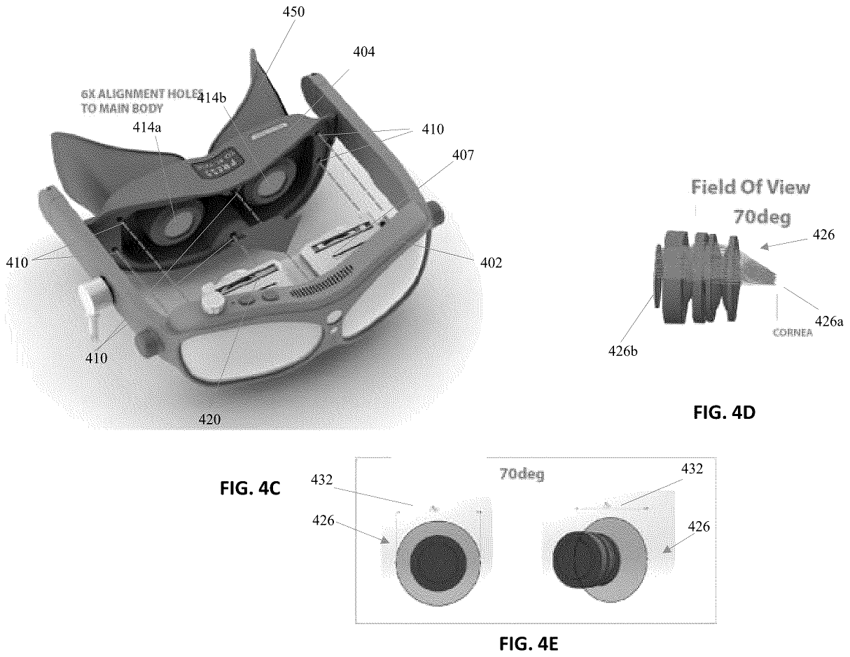

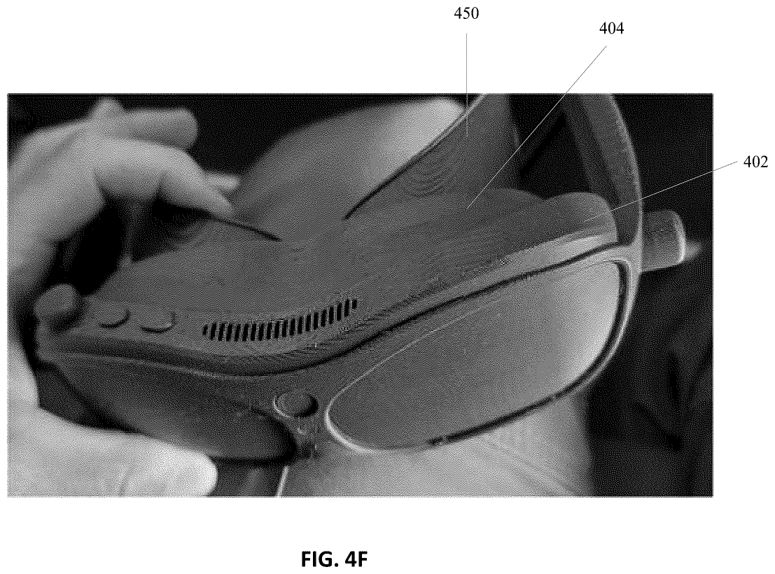

FIG. 4A illustrates a central visual field test module 404 coupled with a host module 402 of vision test device 400, in accordance with an embodiment of the present specification. FIG. 4B illustrates a central visual field test module 404 and the host module 402 of the vision test device, in accordance with an embodiment of the present specification. FIG. 4C illustrates another view of the central visual field test module 404, in accordance with an embodiment of the present specification. FIG. 4D illustrates a plurality of lenses or lens stack 426 of a lens assembly as used with the central visual field test module 404, in accordance with an embodiment of the present specification. FIG. 4E illustrates a diameter of the various lenses that may be used with the central visual field test module, in accordance with an embodiment of the present specification. FIG. 4F illustrates another view of the central visual field test module 404 coupled with the host module 402 of the vision test device 400, in accordance with an embodiment of the present specification. In various embodiments, the central visual field test module 404 is configured to support central visual field assessments of patients, including both threshold and suprathreshold testing. As is known, threshold testing comprises presenting stimuli with varying luminance levels to the patient, in order to determine a luminance level that the patient can detect at least 50% of the number of times the stimuli is presented. Threshold testing enables clinicians to keep track of progressive conditions such as, but not limited to, glaucoma and for quantifying the degree of loss in sensitivity at different visual field test locations.

Luminance is defined as an intensity of emitted light and is measured in units of candelas per meter squared (cd/m²). In an embodiment, for threshold testing, the luminance level of the stimulus on a given trial (presentation of the stimulus together with an interval that allows the user to respond) can vary between background luminance up to the maximum luminance level possible for the vision testing device. The precise luminance level of a stimulus on a given trial depends on how the user responded to that stimulus on previous trials. In embodiments, the presented luminance level of a stimulus is higher than that presented on a previous trial if the user did not detect the previously presented stimulus, and lower if the user detected the previously presented stimulus.

Suprathreshold vision tests comprise presenting high luminance stimuli at each test location to a patient, in order to identify gross visual field defects. In embodiments, for suprathreshold testing, the luminance level of the stimulus either does not vary at all, or varies only between one to three possible levels at any test location. Suprathreshold vision testing is generally faster and suitable for screening purposes, however provides less information about the patient's vision than threshold testing.

Referring to FIGS. 4A, 4B, 4C, and 4D simultaneously, the central visual field test module 404 comprises a first lens assembly 414a and a second lens assembly 414b. In embodiments, first lens assembly 414a and second lens assembly 414b are housed within the central visual field test module 404 adapted to attach with the host module 402. The central visual field test module 404 comprises a button 406 which, when pressed, enables the central visual field test module 404 to be released or disconnected from the host module 402. The central visual field test module 404 also comprises a label 408 identifying the type/function of the module 404, which, in this embodiment is “field test”. The central visual field test module 404 is shaped in order to fit snugly around a patient's eyes. In addition, the central visual field test module 404 comprises two light seals 450, which are used to block out any light from outside of the device. In embodiments, each light seal 450 fits snugly around the perimeter of each of the first lens assembly 414a and second lens assembly 414b. In embodiments, the light seals 450 are attached to the central visual field test module 404 by means of magnetic attachments.

In certain embodiments, the light seals 450 are configured to be removable and interchangeable to accommodate different patient face shapes and anatomical variations. For example, the light seals 450 may be fabricated from compressible foam, elastomeric material, silicone, or other light-blocking material and may be provided in multiple sizes, contours, or thicknesses. In such embodiments, the light seals 450 may be detachably coupled to the central visual field test module 404 via magnetic elements, snap-fit features, or adhesive interfaces, allowing a clinician to select an appropriately contoured light seal 450 to ensure a snug fit around a patient's orbital region and to minimize ambient light intrusion during testing.

In other embodiments, the light seals 450 are uniquely configured for the central visual field test module 404 and are dimensioned to correspond specifically to the geometry of the first lens assembly 414a and second lens assembly 414b and the housing/casework 420 of the host module 402. In such embodiments, the light seals 450 are dedicated components of the central visual field test module 404 and are not interchangeable with light seals of other lens assembly modules, thereby ensuring optimized peripheral light blocking and consistent visual field test conditions.

In embodiments, host module 402 comprises outer housing/casework 420 for covering at least one internal chassis (not visible in FIG. 4B), and preferably a corresponding pair configured to receive each of the first lens assembly 414a and second lens assembly 414b. The casework/housing 420 is also coupled with a first glass layer 422a and a second glass layer 422b, which are coupled with first lens assembly 414a and second lens assembly 414b, respectively, when the central visual field test module 404 is coupled with the host module 402. The distal portion of the central visual field test module 404, which is adapted to attach with the host module 402 comprises six alignment holes/alignment guides 410 provided in an inner region to align with corresponding alignment guides in an inner region of the host module 402. In an embodiment, the alignment guides in an inner region of the host module 402 comprise posts which slide into the holes/alignment guides 410 and, once aligned, the host module 402 and lens assembly module 404 are snap-fit together using magnets positioned on the posts and in the hoes 410. In embodiments, the alignment guides 410 as well as the corresponding alignment guides in an inner region of the host module 402 comprise magnets having opposing polarities, thereby causing the host module 402 and lens assembly module 404 to be attached due to the magnetic force, once aligned.

In an embodiment, each of the first lens assembly 414a and second lens assembly 414b comprise a plurality of lenses 426, shown in FIG. 4D with reference to a cornea of a patient at a proximal end 426a and a display positioned at a distal end 426b, wherein the display is placed proximate to each chassis in the host module 402.

FIG. 4E illustrates an overall diameter of the plurality of lenses 426 of the central visual field test module, in accordance with an embodiment of the present specification. In some embodiments, the plurality of lenses 426 provide a 50 degree to 70 degree field of view and fulfills the standard outer diameter requirement, having a diameter 432 of at least 50 mm when viewed from both the front and the side. In preferred embodiments, the plurality of lenses 426 provide a 70-degree field of view and fulfills the standard outer diameter requirement, having a diameter 432 of 50 mm when viewed from both the front and the side. FIG. 4F illustrates the vision test device 400 comprising the central visual field test module 404 integrated with the host module 402, in accordance with an embodiment of the present specification.

The first lens assembly 414a and second lens assembly 414b are configured for central visual field assessments of a patient, including both threshold and suprathreshold vision testing, and operates within a range of 50° to 80° field of view. The ranges provided here are illustrative and non-limiting.

In various embodiments, threshold tests that may be conducted by using the lens assembly module 404 comprise 24-2, 10-2 and 30-2 test patterns of the Humphrey Field Analyzer. In embodiments, the lens and display parameters of the lens assembly module and host module may be varied depending on which threshold and suprathreshold tests are intended to be administered by using the visual test device. For example, the 60° angular field of view may be increased or decreased based on the farthest extent of the test locations in a corresponding test pattern. In embodiments, Field of View (FOV) is defined by a circle of radius “X”, where “X” is half the diameter of the circle. The displays used in conjunction with the vision test device of the present specification are usually rectangular in shape; however, the device operates with a circular FOV meaning thereby that the circle is inscribed within the rectangular display. In an embodiment, the FOV of the lens assembly module 404 for common test patterns of 24-2 and 30-2 is 60° diameter at minimum. However, in the lens assembly module 404 for the 10-2 test pattern, an FoV of about 20° may be sufficient. In various embodiments the requisite FOV of the lens assembly module 404 depends on the test pattern being displayed to a user.

Similarly, a different type of lens (such as, pancake lens) may be used instead of a fresnel lens if the maximum luminance level needed to measure sensitivity is less than a predetermined threshold level.

In embodiments, a larger field of view of the lens and display system of the vision test device 400 leads to a lower resolution. Hence, the device 400 employs a trade-off between resolution and field of view. In embodiments, the Goldmann III test stimulus, which is a 0.43° diameter spot of light, is used during operation of the device 400. In different embodiments, smaller test stimuli such as the Goldmann I and II as well as larger test stimuli such as the Goldmann IV and V may also be used in conjunction with the device 400. For larger test stimuli, the test resolution may be lower and the field of view may be larger.

The device 400 may be used for monocular testing and may be adapted for binocular testing as well by presenting stimuli to both eyes of a patient simultaneously, instead of to just one eye. In an embodiment, eye tracking of a patient may also be conducted via the device 400.

### Ptosis and Binocular Visual Field Test Module

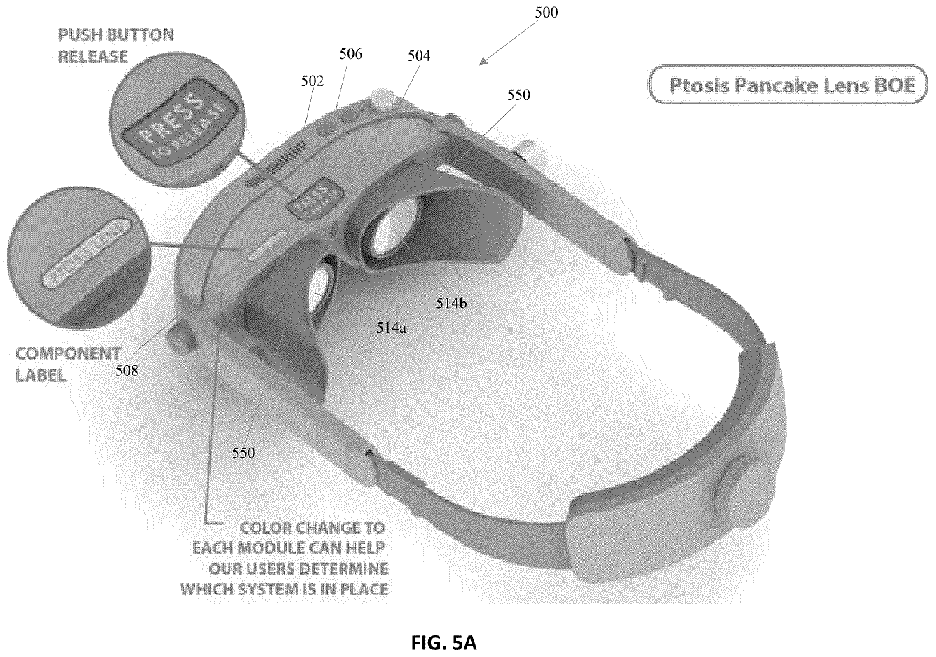

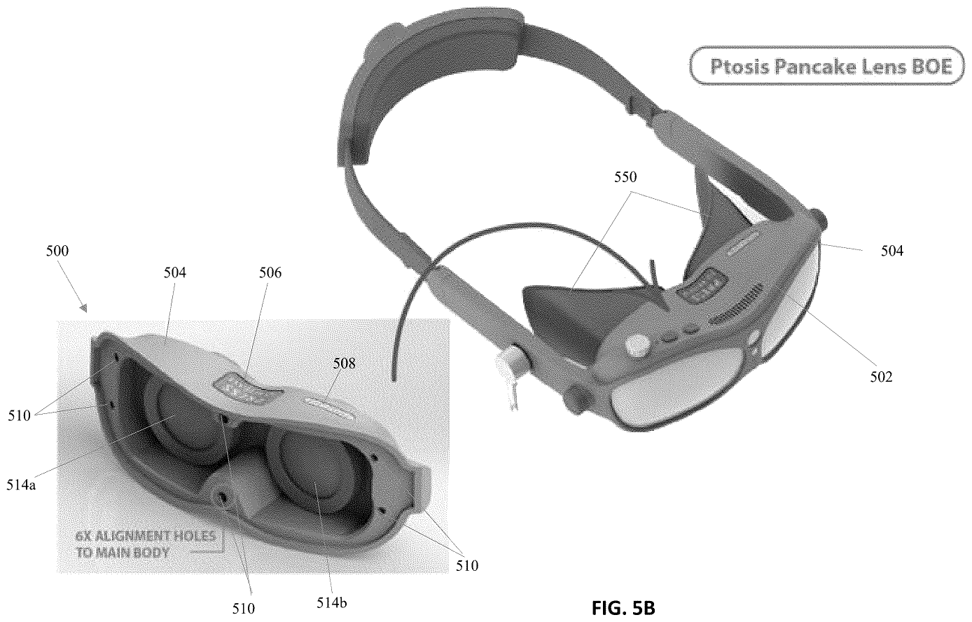

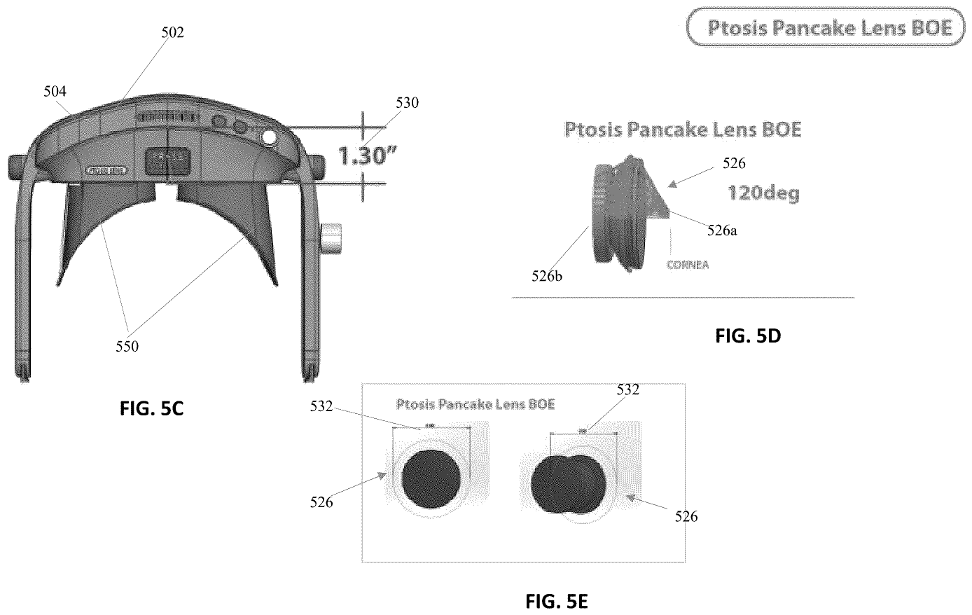

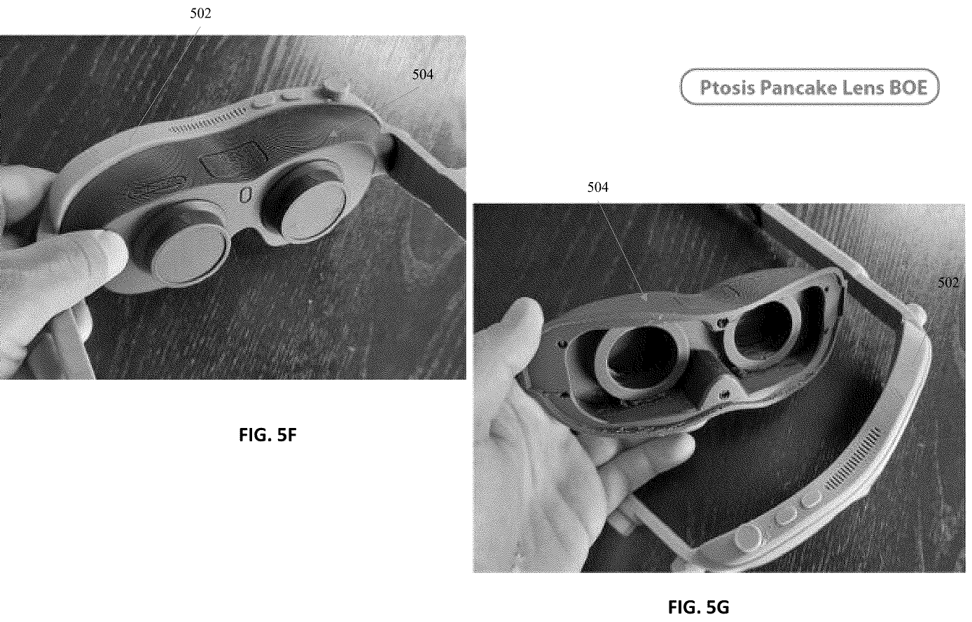

FIG. 5A illustrates a ptosis and binocular visual field test module 504 coupled with the host module 502 of the vision test device, in accordance with an embodiment of the present specification. FIG. 5B illustrates another view of the ptosis and binocular visual field test module coupled with the host module of the vision test device, in accordance with an embodiment of the present specification. FIG. 5C illustrates the width of the ptosis and binocular visual field test module, in accordance with an embodiment of the present specification. FIG. 5D illustrates a lens of a lens assembly of the ptosis and binocular visual field test module, in accordance with an embodiment of the present specification. FIG. 5E illustrates an overall diameter of the plurality of lenses of the ptosis and binocular visual field test module, in accordance with an embodiment of the present specification.

In various embodiments, the ptosis and binocular visual field test module 504 is designed for performing ptosis and binocular visual field testing on patients, supporting applications in scenarios such as, but not limited to, driving, where a large field of view (such as up to 110°) is essential.

The ptosis and binocular visual field test module uses, in embodiments, a pancake lens in order to provide the requisite field of view and minimal form factor. In an embodiment, the lens and display parameters of the ptosis and binocular visual field test module may be modified in order to obtain a required field of view, resolution and maximum luminance for testing patients. In an embodiment, eye tracking of a patient may also be conducted via the ptosis and binocular visual field test module 504. In an embodiment, eye tracking is carried out in order to ensure test accuracy, since the field of view (up to 110°) provided by the ptosis and binocular visual field test module is not as extensive as may be required for performing a visual field test that does not require moving fixation. Hence, reliable test results may be obtained via the module by also tracking a patient's eye movements by ensuring accurate fixation tracking during testing. In an embodiment, the ptosis and binocular visual field test module may move the fixation stimulus provided during testing that require shifting the patient's focus, thereby improving the detection of peripheral defects in the patient's vision.

Referring to FIGS. 5A, 5B, 5C, 5D, and 5E simultaneously, the ptosis and binocular visual field test module 504 comprises a first lens assembly 514a and a second lens assembly 514b. In embodiments, first lens assembly 514a and second lens assembly 514b are housed within an outer frame of the test module 504 which is adapted to attach with the host module 502. The frame of the test module 504 comprises a button 506 which, when pressed, enables the ptosis and binocular visual field test module 504 to be released or disconnected from the host module 502, and a label 508 identifying the type/function of the module 504, which in this embodiment is ‘ptosis lens’. The test module 504 is shaped in order to fit snugly around a patient's eyes. In addition, the ptosis and binocular vision field test module 504 includes two light seals 550, which are used to block out any light from outside of the device. In embodiments, each light seal 550 fits snugly around the perimeter of each of the first lens assembly 514a and second lens assembly 514b. In embodiments, the light seals 550 are attached to the ptosis and binocular vision field test module 504 by means of magnetic attachments.

The distal portion the ptosis and binocular vision field test module 504 which is adapted to attach with the host module 502 comprises six alignment holes/alignment guides 510 provided in an inner region to align with corresponding alignment guides in an inner region of the host module 502. In an embodiment, the alignment guides in an inner region of the host module 502 comprise posts which slide into the holes/alignment guides 510 and, once aligned, the host module 502 and lens assembly module 504 are snap-fit together using magnets positioned on the posts and in the holes/guides 510. In embodiments, the alignment guides 510 as well as the corresponding alignment guides in an inner region of the host module 502 comprise magnets having opposing polarities, thereby causing, the host module 502 and lens assembly module 504 to be attached due to the magnetic force, once aligned.

FIG. 5C illustrates a width or depth 530 of the ptosis and binocular vision field test module 504, in accordance with an embodiment of the present specification. In some embodiments, the width 530 of the ptosis and binocular vision field test module 504 is approximately 0.90 to 1.70 inches from a center point on the device from either side. In a preferred embodiment, the width 530 of the ptosis and binocular vision field test module 504 is approximately 1.30 inches from a center point on the device from either side. In an embodiment, each of the first lens assembly 514a and second lens assembly 514b comprise at least one lens 526, shown in FIG. 5D with reference to a cornea of a patient at a proximal end 526a and a display positioned at a distal end 526b, wherein the display is placed proximate to each chassis in the host module 502. FIG. 5E illustrates an overall diameter of the plurality of lenses 526, in accordance with an embodiment of the present specification. In some embodiments, the at least one lens 526 provides a range of 80 degree to 160 degree field of view and fulfills the standard outer diameter requirement, having a diameter 532 of at least 50 mm when viewed from both the front and side. In preferred embodiments, the at least one lens 526 provides a 120-degree field of view and fulfills the standard outer diameter requirement, having a diameter 532 of 50 mm when viewed from both the front and side.

FIG. 5F illustrates the ptosis and binocular visual field test module 504 attached to the host module 502 of the vision test device 500, in accordance with an embodiment of the present specification. FIG. 5G illustrates the ptosis and binocular visual field test module 504 detached from the host module 502 of the vision test device, in accordance with an embodiment of the present specification.

### Keratometry/Corneal Curvature Measurement Module

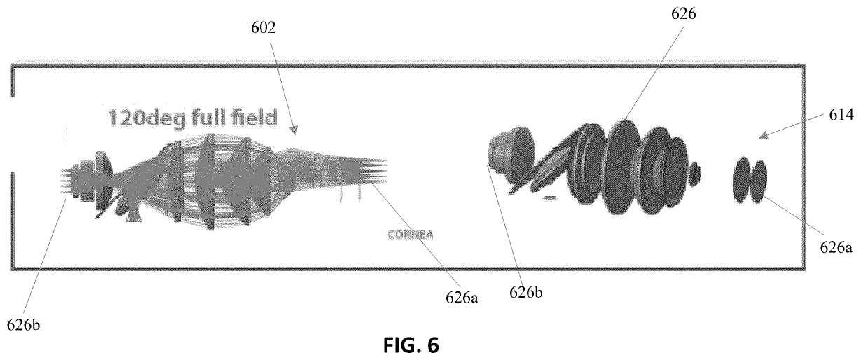

In various embodiments, aa keratometry/corneal curvature measurement module is designed by measuring corneal curvature of a patient, which may determine irregularities indicative of keratoconus. FIG. 6A illustrates a lens assembly 614 of the keratoconus test module. In embodiments, lens assembly 614 comprises a plurality of lenses 626 stacked together having a proximal end 626a which is adapted to be placed proximate a cornea of a patient, and a distal end 626b which is adapted to be placed proximate to a chassis in a host module of the vision test device in which it is used. In embodiments, the plurality of lenses 626 provide a wide field of view, which in certain embodiments, may be an approximately 120 degrees field of view and fulfil the standard outer diameter requirement.

In various embodiments, the keratoconus test module comprises an outer frame and attachment features configured to detachably couple to the host module in a manner consistent with the lens assembly modules described with reference to FIGS. 1A-1G. For example, the keratoconus test module may include alignment guides, magnetic coupling elements, and one or more lens features configured to engage corresponding host module features, thereby enabling secure mechanical attachment, repeatable optical alignment, and automatic module identification by the host module. Accordingly, the keratoconus test module is interchangeable with other lens assembly modules and operable within the same head-mounted vision testing device architecture.

### Vision Test Device and Method of Use

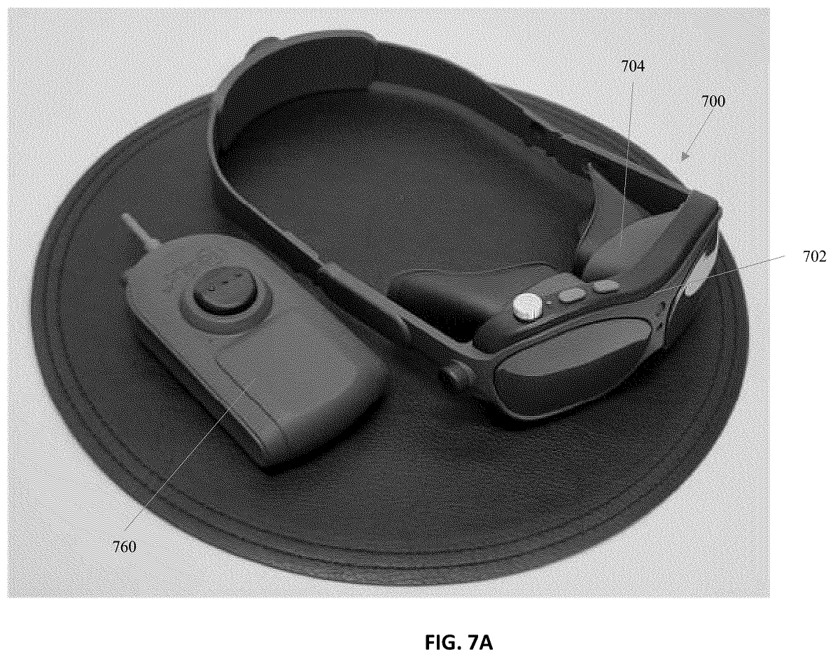

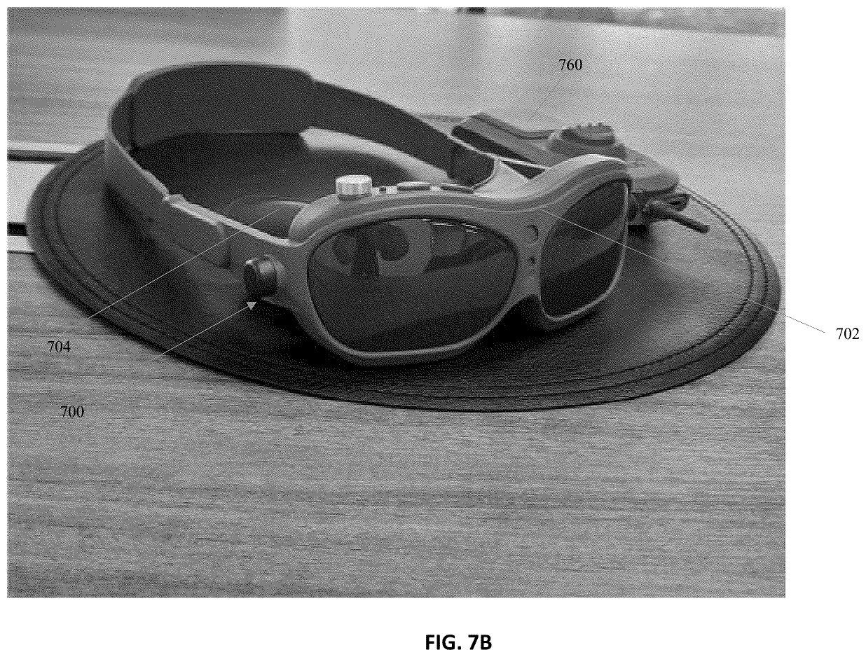

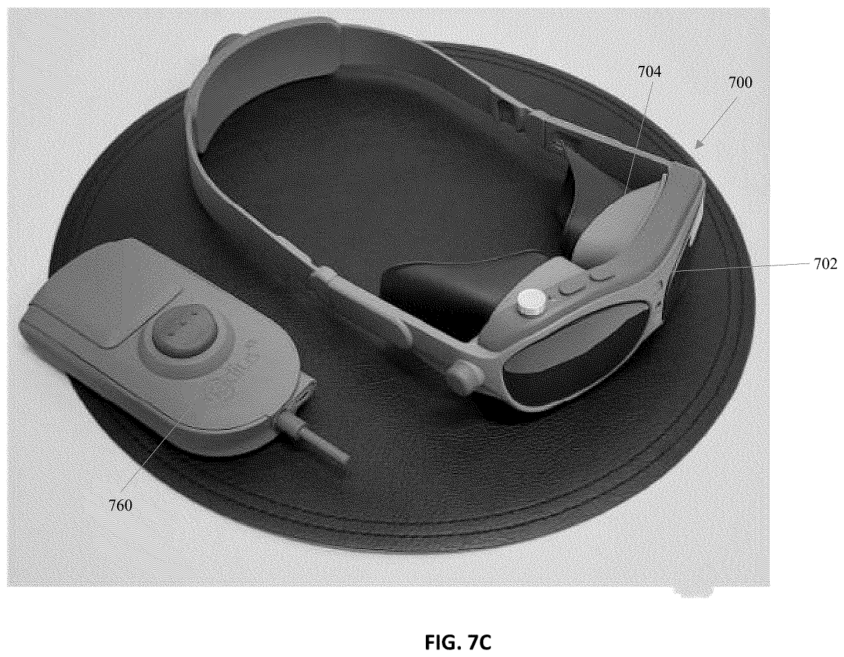

FIG. 7A illustrates a model of the vision test device 700 tethered with a haptic control device 760, in accordance with an embodiment of the present specification. The vision test device 700 is designed as a headset to be worn by a patient/user. The device 700 comprises a host module 702 which is adapted to be coupled with a plurality of lens assembly modules 704 differing in design and functionality as has been explained above. In various embodiments, the host module 702 is equipped with a detection mechanism that identifies which lens assembly module 704 is attached. Once a module 704 is detected, the host module 702 restricts test availability to those compatible with the module 704, thereby preventing inappropriate test selections. This feature enhances the safety and accuracy of diagnostic procedures. In embodiments, the host module 702 integrates a haptic control device 760 tethered to the headset 700, simplifying user interaction and providing power (via a battery pack) to the system. In embodiments, the haptic device 760 serves as a remote control device allowing a user to provide inputs via clicking a button of the device 760, during the vision test. FIG. 7B illustrates another model of the vision test device tethered with a haptic control device, in accordance with an embodiment of the present specification. FIG. 7C illustrates yet another model of the vision test device tethered with a haptic control device 760, in accordance with an embodiment of the present specification.

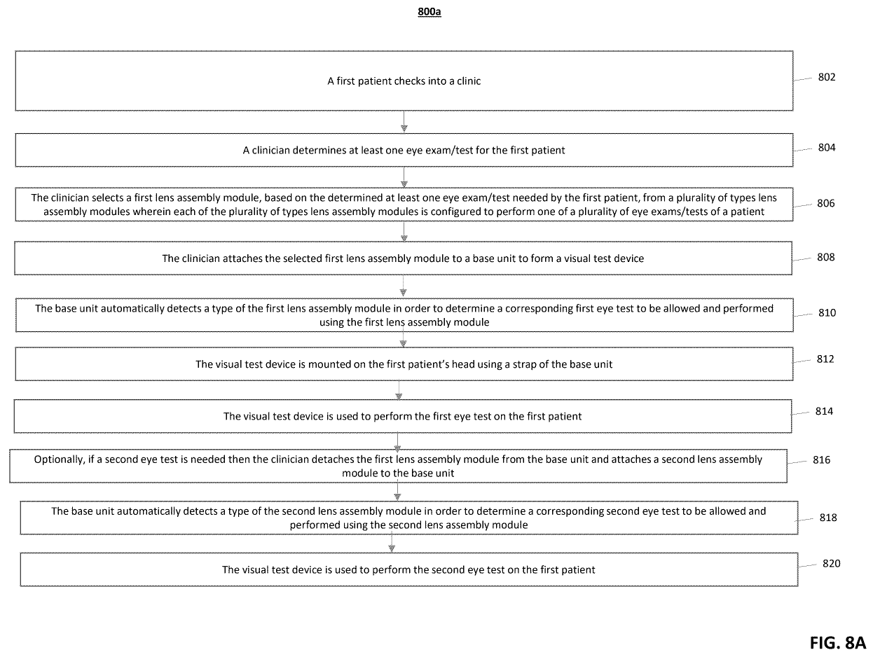

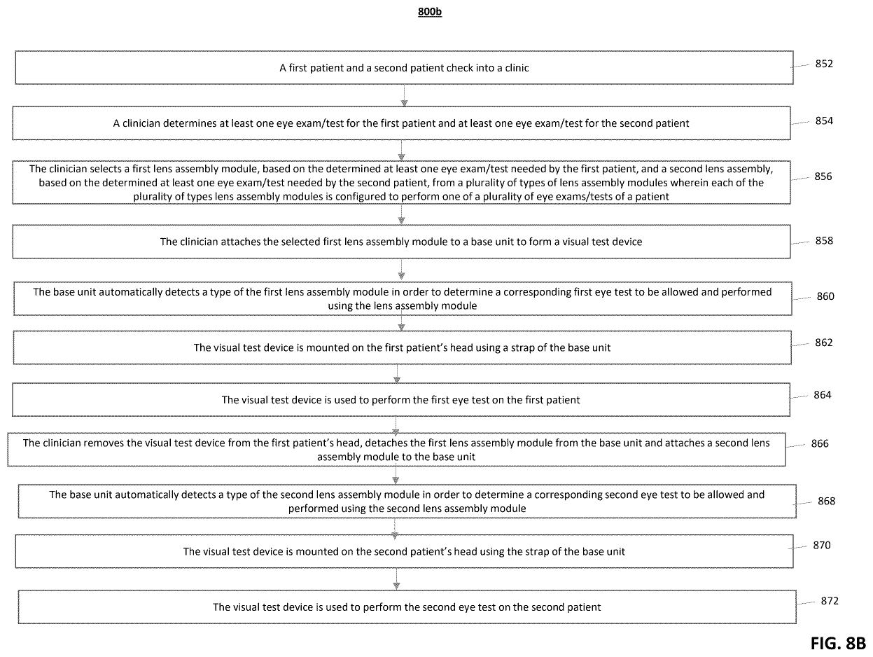

FIG. 8A is a flow chart 800a describing steps of a method for using the visual test device with at least one lens assembly module with one patient, in accordance with some embodiments of the present specification.

At step 802, a first patient checks into a clinic.

At step 804, a clinician determines at least one eye exam/test for the first patient.

At step 806, the clinician selects a first lens assembly module, based on the determined at least one eye exam/test needed by the first patient, from a plurality of types of lens assembly modules wherein each of the plurality of types of lens assembly modules is configured to perform one of a plurality of eye exams/tests of a patient. In various embodiments, the plurality of types of lens assembly modules correspond to a lens assembly module configured to perform a central visual field test, a lens assembly module configured to perform a ptosis and binocular visual field test, a lens assembly module configured to perform a visual acuity and phoropter test, and a lens assembly module configured to perform a keratoconus test.

At step 808, the clinician attaches the selected first lens assembly module to a host module to form a visual test device.

At step 810, the host module automatically detects a type of the first lens assembly module in order to determine a corresponding first eye test to be allowed and performed using the lens assembly module.

At step 812, the visual test device is mounted on the first patient's head using a strap of the host module. In embodiments, the strap includes a knob configured to be rotated to adjust a fit of the visual test device round the first patient's head.

At step 814, the visual test device is used to perform the first eye test on the first patient, wherein the first eye test corresponds to the type of the first lens assembly module determined at step 810.

Optionally, if a second eye test is needed then, at step 816, the clinician detaches the first lens assembly module from the host module and attaches a second lens assembly module to the host module. In embodiments, a button, on an outer frame of the first lens assembly module, is depressed to enable the first lens assembly module to be detached from the host module.

At step 818, the host module automatically detects a type of the second lens assembly module in order to determine a corresponding second eye test to be allowed and performed using the second lens assembly module.

At step 820, the visual test device is used to perform the second eye test on the first patient, wherein the second eye test corresponds to the type of the second lens assembly module determined at step 818.

FIG. 8B is a flow chart 800b describing steps of a method for using the visual test device with at least one lens assembly module switching from a first patient to a second patient, in accordance with some embodiments of the present specification.

At step 852, a first patient and a second patient check into a clinic.

At step 854, a clinician determines at least one eye exam/test for the first patient and at least one eye exam/test for the second patient. In some embodiments, it is determined that the at least one eye exam/test for the first patient is different from the at least one eye exam/test for the second patient.

At step 856, the clinician selects a first lens assembly module, based on the determined at least one eye exam/test needed by the first patient, and a second lens assembly, based on the determined at least one eye exam/test needed by the second patient, from a plurality of types of lens assembly modules wherein each of the plurality of types of lens assembly modules is configured to perform one of a plurality of eye exams/tests of a patient. In various embodiments, the plurality of types of lens assembly modules correspond to a lens assembly module configured to perform a central visual field test, a lens assembly module configured to perform a ptosis and binocular visual field test, a lens assembly module configured to perform a visual acuity and phoropter test, and a lens assembly module configured to perform a keratoconus test.

At step 858, the clinician attaches the selected first lens assembly module to a host module to form a visual test device.

At step 860, the host module automatically detects a type of the first lens assembly module in order to determine a corresponding first eye test to be allowed and performed using the lens assembly module.

At step 862, the visual test device is mounted on the first patient's head using a strap of the host module. In embodiments, the strap includes a knob configured to be rotated to adjust a fit of the visual test device round the first patient's head.

At step 864, the visual test device is used to perform the first eye test on the first patient, wherein the first eye test corresponds to the type of the first lens assembly module determined at step 860.

At step 866, the clinician removes the visual test device from the first patient's head, detaches the first lens assembly module from the host module and attaches a second lens assembly module to the host module. In embodiments, a button, on an outer frame of the first lens assembly module, is depressed to enable the first lens assembly module to be detached from the host module.

At step 868, the host module automatically detects a type of the second lens assembly module in order to determine a corresponding second eye test to be allowed and performed using the second lens assembly module.

At step 870, the visual test device is mounted on the second patient's head using the strap of the host module.

At step 872, the visual test device is used to perform the second eye test on the second patient, wherein the second eye test corresponds to the type of the second lens assembly module determined at step 868.

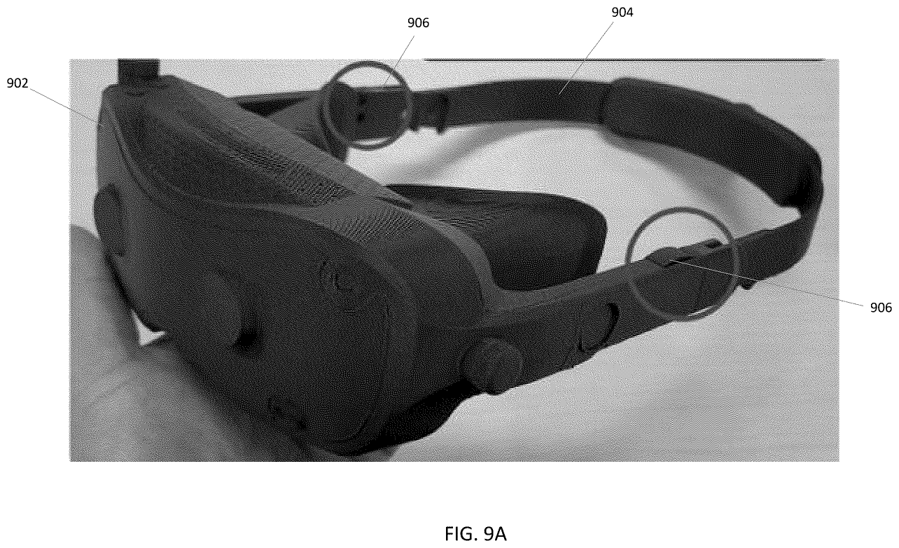

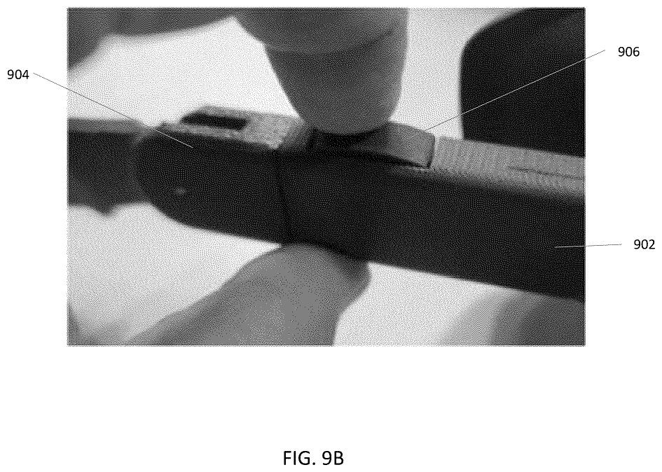

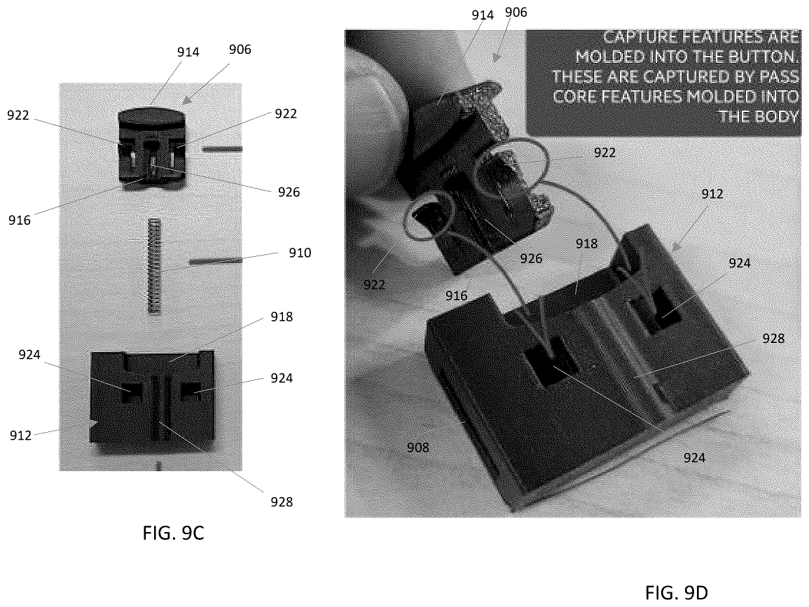

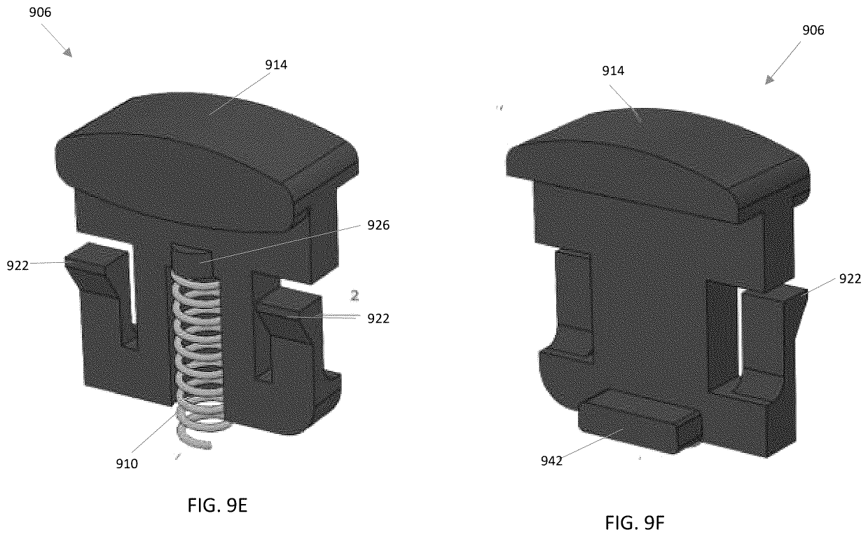

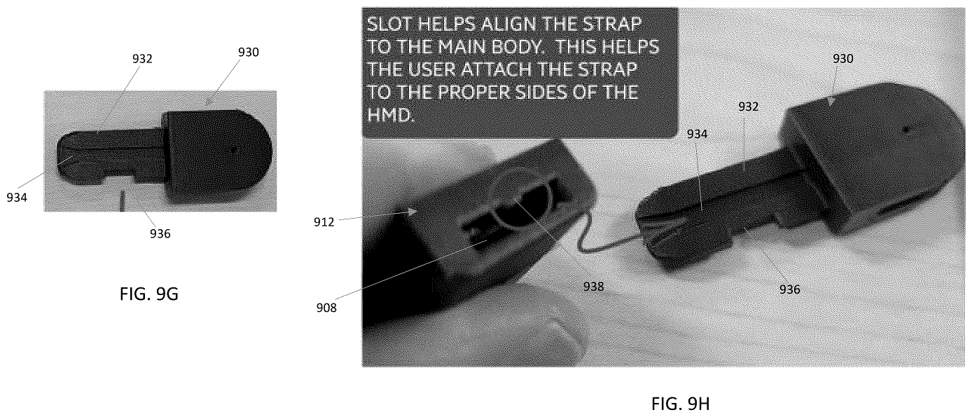

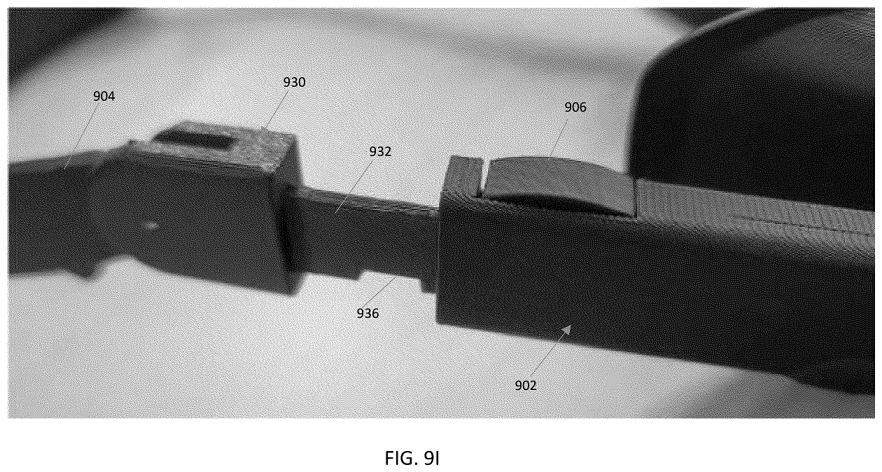

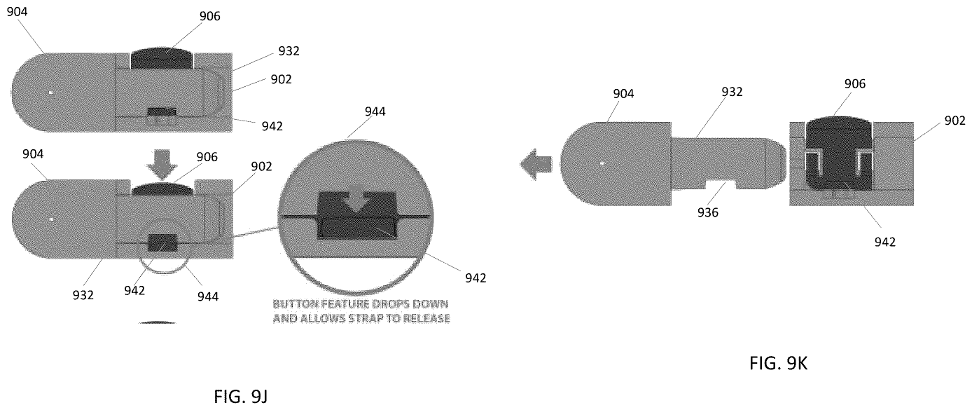

FIG. 9A illustrates the host module/head-mounted device (HMD) coupled with a strap, in accordance with an embodiment of the present specification. As shown in FIG. 9A, in an embodiment, host module 902 is coupled with a strap 904 that is designed to fit around a patient/user's head. The host module 902 comprises at least one release button 906 provided on each side of the host module 902, which when pressed releases/detaches the strap 904 from the host module 902. FIG. 9B illustrates the strap being released from the body of the host module by pressing the button, in accordance with an embodiment of the present specification. FIG. 9C illustrates the button, a spring, and a portion of the body of the host module that houses the button and the spring, in accordance with an embodiment of the present specification. FIG. 9D illustrates a close up view of the button and the portion of the body of the host module that houses the button, in accordance with an embodiment of the present specification. FIG. 9E illustrates a pictorial side plan view of a first side of the button, in accordance with an embodiment of the present specification. FIG. 9F is a pictorial side plan view of a second side of the button, wherein the second side is positioned opposite to the first side, in accordance with an embodiment of the present specification.

Referring to FIGS. 9A through 9F, simultaneously, the body of the host module 902 comprises a slot 908 for receiving a portion of the strap 904. Upon pressing the button 906, said portion is released/detached from the body of the host module 902.

The button 906 and a spring 910 are designed to fit into a molded portion 912 of the body of the host module 902. In an embodiment, the button 906 is designed as a rectangular piece having a dimension or size that can accommodate the length, width and thickness of the strap and host module. A proximal end 914 of the button 906 comprises an outward and protruding curved section that forms a portion of the button 906 that is pressed by a user for releasing the strap 904 from the host module 902 as is shown in FIG. 9B. A distal end 916 of the button 906 is inserted within a slot 918 of the molded portion 912 such that the entire length of the button 902 is seated within the slot 918, and only the outward curved portion at the proximal end 914 protrudes partially outward from the slot 918. Each side of the length of the rectangular button 906 comprises a capture feature 922 which protrudes outwards from a first surface/side of the button 906. Each capture feature 922 is designed to fit into a core feature 924 molded into the portion 912 for ensuring that the button 906 is securely held within the slot 918 of the molded portion 912. In an embodiment, each of the core feature 924 is designed as a slot sized to securely hold the capture feature 922.

The body of the button 906 further comprises an elongated groove 926 positioned between the two capture features 922 on the first surface/side of the button 906, as also shown in FIG. 9E. The molded portion 912 of the body of the host module 902 also comprises an elongated groove 928 positioned between the two core features 924. The spring 910 is seated within the groove 926 and groove 928 when the button 906 is inserted into the slot 918. When the button 906 is not depressed, the spring 910 is in a decompressed state, creating a tension fit with the strap attachment portion, as described below.

An additional capture feature 942 is positioned near the distal end 916 and protrudes outwards from a second surface/side opposite to the first surface/side of the body of the button 906. The second opposing surface/side of the button 906 comprising the capture feature 942 protruding outwards from the second surface/side is illustrated in FIG. 9F.

FIG. 9G illustrates an attachment portion of the strap, in accordance with an embodiment of the present specification. FIG. 9H illustrates the attachment portion of the strap and a slot provided in the body of the host module for receiving the attachment portion, in accordance with an embodiment of the present specification. FIG. 9I illustrates the attachment portion of the strap partially inserted into the slot provided in the body of the host module, in accordance with an embodiment of the present specification.

Referring to FIGS. 9A-9I attachment portion 930 of the strap 904 comprises a thin elongated portion 932 designed to fit into the slot 908 provided in the molded portion 912 for receiving said elongate portion 932, which further comprises an elongate groove 934 and a capture feature 936, as shown in FIG. 9G. The slot 908 comprises an elongate protruding feature 938 designed to fit into the groove 934 when the elongated portion 932 is inserted into the slot 908, thereby securing the strap to the body of the host module 902. The capture feature 936 engages with the feature 942 of the button 906 when the strap 904 is inserted into the slot 908. The design of the capture feature 936 ensures that a first predefined side of the strap 904 is attached with a first predefined side of the host module 902. The capture feature 936 enables a user to attach the strap 904 correctly to the host module 902 by providing an alignment guide.

FIG. 9J illustrates a sectional view of the strap attached to the body of the host module, in accordance with an embodiment of the present specification. FIG. 9K illustrates a sectional view of the strap being released from the body of the host module, in accordance with an embodiment of the present specification. Referring to FIGS. 9A-9K, and specifically as shown in FIG. 9J, the elongated portion 932 of strap 904 inserted into slot 908 is positioned such that the feature 942 of the button 906 engages with the capture feature 936 for securely holding the strap portion 932 within the slot 908, thereby attaching the strap 904 to the body of the host module 902. When the button 906 is pressed, the spring 910 securely enclosed within the grooves 926 and 928, is decompressed, thereby providing a spring-loaded button release system for causing the strap 904 to be released. The depression of the button 906 causes the feature 942 of the button 906 to drop down (as can be seen in the magnified view 944), resulting in a release of the elongate portion 932 of strap 904. Once the portion 942 lowers due to the button 906 being pressed, the elongate portion 932 is no longer held within the slot 908 and can be withdrawn from the body of the host module 902 as shown in FIG. 9K.

The above examples are merely illustrative of the many applications of the systems and methods of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A head-mounted vision test device comprising:
a host module, comprising a processing unit and a display; and
a lens assembly module, wherein the lens assembly module is adapted to be detachably connected to the host module and wherein the host module is configured to automatically detect and identify a type of a lens assembly module that is connected to the host module, wherein the lens assembly module comprises an outer frame and at least one lens assembly comprising at least one lens adapted for testing one of either a visual field of a patient or a visual acuity of the patient, and wherein the host module is configured to execute only one or more vision tests that are uniquely supported by the identified lens assembly module.

2. The head-mounted vision test device of claim 1, wherein the outer frame comprises a button which, when depressed, releases the lens assembly module from the host module.

3. The head-mounted vision test device of claim 1, wherein the outer frame comprises a label indicative of the type of the lens assembly module.

4. The head-mounted vision test device of claim 1, wherein the lens assembly further comprises at least one light block.

5. The head-mounted vision test device of claim 1, wherein the host module comprises a strap to fit around the patient's head while wearing the head-mounted vision test device and wherein the strap includes a knob configured to be rotated to adjust a fit of the strap round the patient's head.

6. The head-mounted vision test device of claim 1, wherein an inner region of the outer frame comprises a first plurality of alignment guides to align with a corresponding second plurality of alignment guides in an inner region of the host module, and wherein the host module is attached to the lens assembly module using a plurality of magnets once the first plurality of alignment guides aligns with the corresponding second plurality of alignment guides.

7. The head-mounted vision test device of claim 1, wherein the host module comprises a first internal chassis including a first host module feature and a second internal chassis including a second host module feature, wherein the lens assembly module comprises a first lens assembly including a first lens feature and a second lens assembly including a second lens feature, and wherein the first host module feature is configured to receive the first lens feature and the second host module feature is configured to receive the second lens feature to enable the lens assembly to be detachably connected to the host module.

8. The head-mounted vision test device of claim 7, wherein the first and second internal chassis are covered by an outer housing, and wherein the outer housing includes a first glass layer and a second glass layer for respectively coupling with the first lens assembly and the second lens assembly.

9. The head-mounted vision test device of claim 8, wherein each of the first and second glass layers is fabricated from fusilica, gorilla glass, or BK7.

10. The head-mounted vision test device of claim 1, wherein the lens assembly module is configured for one of a central visual field test, a ptosis and binocular visual field test, a visual acuity test, a phoropter test or a keratoconus test.

11. The head-mounted vision test device of claim 1, wherein the lens assembly module is configured for a visual acuity and phoropter test, and wherein the lens assembly module comprises a plurality of lenses that provide a 14 degrees field of view.

12. The head-mounted vision test device of claim 1, wherein the lens assembly module is configured for a central visual field test including threshold and suprathreshold testing.

13. The head-mounted vision test device of claim 12, wherein the lens assembly module operates within a 60° field of view.

14. The head-mounted vision test device of claim 1, wherein the lens assembly module is configured for a central visual field test including monocular and binocular vision testing.

15. The head-mounted vision test device of claim 1, wherein the lens assembly module is configured for a ptosis and binocular visual field test.

16. The head-mounted vision test device of claim 15, wherein the lens assembly module operates within a 120° field of view.

17. The head-mounted vision test device of claim 15, wherein the lens assembly module comprises one or more pancake lenses.

18. The head-mounted vision test device of claim 1, wherein the lens assembly module is configured for a keratometry test by measuring corneal curvature of a patient.

19. The head-mounted vision test device of claim 1, further comprising a remote control device tethered to the head-mounted vision test device.

20. The head-mounted vision test device of claim 19, wherein the remote control device comprises a battery for powering the head-mounted vision test device.

21. The head-mounted vision test device of claim 1, wherein the host module comprises means for tracking an eye movement of the user.

22. The head-mounted vision test device of claim 1, wherein the lens assembly module comprises optically distinct lens stacks configured to optimize at least one of a field of view, an angular resolution, a luminance transmission, and an optical magnification for a corresponding diagnosis.

23. The head-mounted vision test device of claim 22, wherein each lens stack is structurally configured for a specific diagnostic test selected from visual acuity testing, visual field testing, binocular field assessment, or corneal curvature assessment.

24. The head-mounted vision test device of claim 1, wherein the host module is further configured to disable a selection of one or more diagnostic tests that are incompatible with optical characteristics of the identified lens assembly module.
